(19) United States
(12) Patent Application Publication
(10) Pub. No.: US 2021/0247386 A1
(43) Pub. Date: Aug. 12, 2021

(54) A DEVICE AND METHOD FOR DETERMINING CELL INDENTATION ACTIVITY

(71) Applicant: TECHNION RESEARCH & DEVELOPMENT FOUNDATION LIMITED, Haifa (IL)

(72) Inventors: Daphne WEIHS, Haifa (IL); Yulia MERKHER, Kiryat Tivon (IL)

(21) Appl. No.: 17/050,560

(22) PCT Filed: Apr. 24, 2019
(86) PCT No.: PCT/IL2019/050463

§ 371 Date: Oct. 26, 2020

Related U.S. Application Data
(60) Provisional application No. 62/662,866, filed on Apr. 26, 2018.

(51) Int. Cl. G01N 33/50

(52) U.S. Cl. CPC G01N 33/5091; G01N 2500/10; G01N 33/5011

(57) ABSTRACT

The present invention is direct to methods for determining cell indentation activity, and diagnosis or prognosis of cancer. Further provided is a device comprising a gel having a Young's modulus of 0.1-20 kPa.

### CROSS REFERENCE TO RELATED APPLICATIONS

This application claims the benefit of priority to U.S. Provisional Patent Application No. 62/662,866 titled “A DEVICE AND METHOD FOR DETERMINING CELL INDENTATION ACTIVITY”, filed Apr. 26, 2018, the contents of which is incorporated herein by reference in its entirety.

### FIELD OF INVENTION

The present invention is in the field of mechanobiology.

### BACKGROUND OF THE INVENTION

The major cause of cancer-related deaths is due to metastasis—the spread of tumor cells to other organs. Metastasis diagnosis and prognosis is currently based on shape/size classification of a resected tumor including histology, on lymph node status (giving as many as 30% false negatives) or on tumor genetics. Existing predictors are not infallible even in cases where genetic markers have been identified. Furthermore, genetic testing is costly, and limited with some prognostic markers known to fail primarily to due low sensitivity or specificity. Importantly, none of the currently existing approaches are fully reliable, or provide diagnosis and prognosis during or close to the time of the initial surgery, and in some cases delivering of diagnosis/prognosis to patients can take as many as few weeks. In addition, currently practiced methods rely on generic knowledge for drug selection rather than on a tailored patient-personalized approach.

### SUMMARY OF THE INVENTION

The present invention is directed to a method for determining indentation activity of cells. In some embodiments, the method is directed to diagnosis or prognosis of cancer in a subject. Further provided is a device comprising a gel having a stiffness of 0.1-20 kPa.

According to one aspect, there is provided a method of determining indentation activity of a cell population, the method comprising: contacting the cell population with a gel having a Young's modulus of 0.1-20 kPa; and measuring a cell indentation parameter using at least one sensor responsive to signals ranging between 1 mPa-20 kPa, wherein an increase in at least one cell indentation parameter relative to control, is indicative of increased indentation activity of the cell population.

According to another aspect, there is provided a method of classifying a cell population according to indentation activity, the method comprising: contacting a cell population with a first gel having a Young's modulus of 0.1-20 kPa; measuring a cell indentation parameter, thereby determining the cell population indentation activity; and determining a cell characteristic of the cell population based on a pre-determined indentation activity threshold, wherein the cell characteristic is selected from the group consisting of: invasiveness, metastatic potential, infiltration, and differentiation state, thereby classifying the cell population according to the indentation activity.

According to another aspect, there is provided a computer program product for determining cell indentation activity, the computer program product comprising a non-transitory computer-readable storage medium having program instructions embodied therewith, the program instructions executable by at least one hardware processor to: receive measurements of at least one cell indentation parameter of a cell population contacted with a gel having a Young's modulus of 0.1-20 kPa; and determine a cell characteristic of the cell population based on, at least in part, a pre-determined indentation activity threshold, wherein the cell characteristic is selected from the group consisting of: invasiveness, metastatic potential, infiltration, and differentiation state.

According to another aspect, there is provided a device comprising: a gel having a Young's modulus of 0.1-20 kPa; and at least one sensor responsive to signals ranging between 1 mPa-20 kPa, in contact with the gel.

In some embodiments, the indentation activity parameter comprises the number of indenting cells, the indentation depth attained by the cells, the force applied by the cells to the gel, the pressure applied by the cells to the gel, the strain applied by the cells to the gel, the displacement applied by the cells to the gel, or any combination thereof.

In some embodiments, the cell population is obtained from a sample being obtained from a subject.

In some embodiments, the method is for diagnosing cancer in a subject, wherein increased indentation activity of the cell population relative to control is indicative of cancer in the subject.

In some embodiments, the method further comprises a step of quantifying the cell population indentation activity, wherein increased indentation activity of the cell population relative to control is a prediction or prognosis of metastatic cancer in the subject.

In some embodiments, the prediction of the metastatic cancer comprises predicting the target organ for metastases by further comparing the indentation activity of the cell population on a second gel having a different stiffness compared to the first gel.

In some embodiments, the method is for screening for a compound suitable for reducing indentation activity of the cell population, the method comprising contacting the cell population with the compound, wherein reduction of indentation activity of the cell population in the presence of the compound compared to the indentation activity of the cell population in the absence of the compound indicates the compound is suitable for reducing indentation activity of the cell population.

In some embodiments, the cell population is contacted with the compound prior to contact with the gel, after contact with the gel, or both.

In some embodiments, a compound suitable for reducing indentation activity of a cell is suitable for preventing or reducing cancer invasiveness.

In some embodiments, the measuring comprises the use of a sensor, wherein the sensor is selected from the group consisting of: a pressure sensor, a strain sensor, and an optical sensor. In some embodiments, the sensor is selected from a pressure sensor or a strain sensor.

In some embodiments, the device further comprises an optical sensor.

In some embodiments, the gel further comprises particles. In some embodiments, the particles are fluorescent particles. In some embodiments, the particles are 10 nm to 450 nm in diameter.

In some embodiments, the cell is an infiltrating cell. In some embodiments, the infiltrating cell is a proliferating cell. In some embodiments, the proliferating cell is a cancer cell. In some embodiments, the cancer cell is a metastatic cancer cell. In some embodiments, the cancer cell is a locally invasive cancer cell.

In some embodiments, the increased indentation activity of the cell population relative to control is an indication of cancer, or a prediction or prognosis of metastatic cancer, in the subject.

In some embodiments, the gel is a hydrogel comprising at least 50% water by weight. In some embodiments, the gel comprises at least one biologically inert polymer. In some embodiments, the gel is impenetrable to a cell. In some embodiments, the gel comprises pores, wherein at least 80% of said pores have a diameter of between 1 and 500 nm. In some embodiments, the gel has a thickness of 30-500 μm.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1F are images and micrographs of a non-limiting exemplary process of cell extraction from tissue sample. (1A) Resected tumor samples in Histidine-tryptophan-ketoglutarate (HTK) preservation solution are transported to the lab at 4° C. (1B) Samples are measured and minced and cells are isolated by enzymatic degradation at 37° C. within 2 hours. Cell and other debris are removed by passing through a 100 μm cell-strainer and (1C) by cell lysis buffer treatment. (1D) Collected cells are seeded on gels (1E) and indentation activity is recorded by imaging (1F).

FIGS. 2A-2E are an illustration and micrographs depicting indentation activity of a cell. (2A) is a side-view sketch of a cell indenting a gel with fluorescent, 200-nm diameter particles embedded in or at its surface. (2B) is a side-view micrograph of fixated, indenting adjacent MDA-MB-231 high metastatic potential cells on polyacrylamide (PAM) gel having a Young's modulus of 2.4 kPa. Image was taken using a confocal microscope; scale bar represents 20 μm. Nuclei are stained (bright blobs) and particles pushed underneath the cells show that each cell had induced different indentation depth below the initially flat, gel surface. (2C-2E) are top-view micrographs taken with fluorescence and light microscopy at the representative heights indicated in FIG. 2A (i.e., cell height, gel surface and gel indent). (2C) is a differential interference contrast (DIC) image of cells seeded on 2.4 kPa gel and exhibiting varying morphologies. (2D) is a fluorescent micrograph of the particles at the gel-surface height (0 μm). Particles underneath many of the cells are out of focus, indicating the subpopulation of cells that have indented the gel (i.e., particles were displaced to a lower focal plane). (2E) is a fluorescent micrograph at 6 μm below the gel-surface height. Particles viewed in focus at that depth (arrows) indicate the indentation depth (i.e., ΔL in FIG. 2A), attained by those cells. Scale bar represents 20 μm.

FIG. 3 is a graph delineating the correlation between physicochemical compositions of PAM gels that can be used, to provide specific Young's modulus for cell indentation studies. The experimental results (▪) are fit to a power trend-line giving Young's modulus=298·α^{-0.91} (solid line), where the parameter α is a newly defined combination of the concentrations of the BIS-acrylamide, [BIS], and acrylamide, [ACR], monomers. The Young's modulus of the experimental results were measured by rheometry. The experimental results were further compared to compositions used by others, where the Young's modulus was determined by various methods, e.g. ball indentation. Traction (lateral) force microscopy studies of metastatic breast, pancreas and lung cancer cells (◯) were adapted from (Ambrosi et al., 2009; Califano and Reinhart-King 2012); no indentation was observed in those studies, possibly due to erroneous stiffness measurements, as exemplified hereinbelow (see example 5). Non-invasive cells were separately grouped (Δ), fibroblasts and endothelial cells (Tony Yeung et al., 2005), myoblasts (Engler et al., 2004), and mesenchymal stem cells (Flanagan et al., 2002). (⋄) depicts data from a negative control gel (Geissler and Hecht 1981).

FIG. 4 is a graph delineating the correlation between different parameters of cell indentation activity—average indentation depth (μm) and average percentage of indenting cells (%) in a cell population, used as a diagnostic/prognostic plot. Breast (Δ) and pancreatic (□) cancer cell lines and resected pancreatic tumors (◯) were tested on PAM gels with Young's modulus of 2.4 kPa. Proposed cutoff ranges (dashed lines) distinguish non-invasive/benign (‘non-invasive/benign box’ at bottom left) from invasive (‘invasiveness line’) comprising both low invasive (outside box and under the line) and highly invasive cells (over the line). Error bars are standard errors.

FIG. 5 is a graph delineating the correlation between different parameters of cell indentation activity—indentation depth (μm) and distribution of indenting cells (%), of breast (▴) and pancreatic (▪) cancer cell lines before (full markers) and after treatment with Taxol (25 μM for 1 hr, empty markers) tested on PAM gels having Young's modulus of 2.4 kPa. Treatment reduced the number of indenting cells and also the attained depths. All treated cell lines were reduced below the nearest, proposed cutoff. That is, initially highly invasive cells moved under the invasiveness line, and initially less invasive cells moved closer to the non-invasive/benign box. Error bars are standard errors.

FIG. 6 is a graph delineating the correlation between different parameters of cell indentation activity—indentation depth (μm) and distribution of indenting cells (%), showing cancers from different organs may require different gel stiffness for accurate diagnosis/prognosis. Arrows show change in location on diagnostic/prognostic plot of breast (▴) and pancreatic (▪) cancer cell lines moved from 2.4 kPa PAM gels (full markers) to 1.2 kPa gels (empty markers). Reducing the substrate stiffness provided improved resolution for pancreatic cancer while maintaining the same prognostic conclusion on the invasiveness and metastatic potential of breast cancer; the cells remained above/below the prognostic line and non-invasive primary-site pancreatic cancers were essentially un-affected.

FIGS. 7A-7B are images of a FE-bio simulation of 9 widely spaced, cylinders (height 20 μm and diameter 12 μm) each indenting to a depth of 6 μm on a gel-section of size 300×300 μm². The gel and cylinders are modeled as having Young's modulus of 2.4 kPa and 25 kPa, respectively, and both have a Poisson ratio of 0.49. (7A) A map of the z-direction principle stresses; (7B) A map of the z-direction principle strains. Average z-direction stress and strain are measured in an area at the bottom of the gel, underneath the cylinder locations, sized 120×120 μm². Averaged over a gel thickness of 8 μm at the bottom layer the mechanical stress is 88 Pa (translated to a force of 11.6×10⁻⁶N on the 120×120 μm² surface) and the displacement at that location is 0.01878 μM.

FIGS. 8A-8E are graphs describing different mechanical invasiveness measurements and parameters. The mechanical invasiveness is indicated by the cell indentation activity as demonstrated by the average indentation depth (μm) and average percentage of indenting cells (%). (8A) is a graph comprising more samples as presented in FIG. 4, showing mechanical invasiveness measure in pancreatic (▪) and breast (▴) cancer cell lines and freshly resected human pancreatic (●) samples on 2.4 kPa PAM gels. *4 indicates 4 non-indenting normal samples from tumor-adjacent sites in different pancreatic cancer patients. Samples on bottom left are benign/non-invasive and higher samples are cancerous and typically metastatic. Bars are standard errors. (8B) is a graph showing an automated k-means clustering analysis by Euclidian distances performed with results from cell lines (pancreatic, breast) and histopathologically verified clinical samples (including pancreatic normal, precancerous and tumor cells) in FIG. 8A. Confidence interval ellipses show that clusters are statistically distinguished from one another; ellipses are regions containing 50, 70, 90, 95% of the data points. (8C) is a graph showing mechanical invasiveness of fresh skin and stomach cancer samples. Histopathologically confirmed cancer samples (report was obtained at least 4 weeks after sample testing) from subjects, included: basal cell carcinoma (BCC; o) and squamous cell carcinoma (SCC; A). Samples were classified as non-invasive (empty symbols) or invasive (full symbols) by histopathological examination (in BCC indenting-invasive sample has desmoplasia) as compared to control scar sample (□). Freshly resected stomach cancer sample (♦). (8D) is a graph describing the identification of target metastatic sites in the body of pancreatic cell lines. The change in mechanical invasiveness of the well-established pancreatic cancer cell lines, so of which were demonstrated in FIG. 6, was collected from primary site and from metastatic sites on soft (i.e., 1.2 kPa) vs. stiffer (2.4 kPa) gel (represented as percent difference in percent of indenting cells between soft and stiffer gel) correlated with the stiffness of the target site (i.e., spleen, liver, and ascites) as determined from the literature with mechanical measurements of organs inflicted with metastatic cancer (Sakai et al., (2016); Rice et al., (2017); and Ma et al., (2016)). Metastases containing organs are typically stiffer than the same, normal organs. As the established cell lines used in 8D are from metastatic sites, the values shown for the organs are those typically measured at organs that already include metastasis. (8E) is a graph showing the percent of cells, from established breast and pancreatic cancer lines, which trespass a Boyden chamber membrane with 8 μm sized pores; Boyden chambers are considered a common (gold-standard) approach to evaluate in vitro metastatic potential of cells. Cells were serum-starved for 24 hr and then allowed to trespass for 72 hr, according to the standard practice in such experiments. Results of the Boyden chambers analysis are in accordance with the mechanical invasiveness measure, where the latter also provided increased resolution to identify small changes in levels of metastatic potential in the pancreatic cancer cells.

FIGS. 9A-9B are graphs showing the differential PAM gel stiffness value obtained by rheology as compared to ball indentation methodology. (9A) Stiffness of the disclosed PAM hydrogel was measured by rheometer measurements vs. stiffness of gels with the same composition measured by ball indentation method, as described and measured in (Kraning-Rush et al., 2012). (9B) is a graph showing the ratio of gel stiffness value obtained by rheology compared to the ball indentation methodology, of FIG. 9A. Error bars are standard deviations. Text boxes are gels composition of Acryl (% w/v)/BIS (% w/v) matching the recipes provided in (Kraning-Rush et al., 2012).

FIGS. 10A-10F are micrographs of high metastatic potential breast cancer cells from MDA-MB-231 cell line, indenting a 1.2 kPa gel, whereas the sample is imaged at the same location under different magnifications. Scale bars=40 μm. (10A-10C) Differential interference contrast under ×20 (10A), ×40 (10B), and ×60 (10C) magnifications. (10D-10F) Fluorescence microscopy focused on the fluorescent 200-nm particles embedded at the gel's surface under ×20 (10D), ×40 (10E), and ×60 (10F) magnifications.

FIG. 11 is a graph showing the effects of objective lens magnification (20, 40, 60×) on ability to identify indenting cells. The percent of high metastatic potential breast cancer cells (MDA-MB-231) were determined and calculated at the same location on 1.2 kPa PAM gel under different magnifications. Error bars are standard deviations.

FIGS. 12A-12N are fluorescent micrographs demonstrating the effects of objective lens magnification, using 0.2 μm beads embedded at and in the surface of a 5 kPa PAM gel. The images performed at focal heights of 0 μm (12A), −1 μm (12B), −2 μm (12C), −3 μm (12D), −4 μm (12E), −5 μm (12F), −6 μm (12G), +0.5 μm (12H), −1.2 μm (12I), −2.6 μm (12J), −3.6 μm (12K), −4.4 μm (12L), −5.3 μm (12M), and −5.8 μm (12N) under different magnifications (12A-12G: ×20, automated focal height setting; and 12H-12N: ×60, manual focal height setting). Scale bars: 12A-12G=60 μm; 12H-12N=20 μm.

FIGS. 13A-13H are representative micrographs obtained with fluorescent microscope, demonstrating effects of particle size embedded in PAM gels. High metastatic potential breast cancer cells (MDA-MB-231) indenting a 2.4 kPa PAM gel with 500 nm fluorescent beads embedded at its surface (13A). The images in the following panels were performed at the same horizontal location as (13A) and at varying focal heights of 0 μm (13B), −1.57 μm (13C), −2.5 μm (13D), −3.38 μm (13E), −4.59 μm (13F), −5.18 μm (13G), and −6.15 μm (13H). Scale bar=20 μm. Arrows point to indenting cells at each lowest focal depth of focus, i.e. demonstrating their attained indentation depth.

### DETAILED DESCRIPTION OF THE INVENTION

The present invention is directed to a method, a device, and a kit for determining indentation activity of infiltrating cells.

In some embodiments, the invention provides a device comprising a gel having a Young's modulus of 0.1-20 kPa, and at least one sensor responsive to mechanical stress signals ranging between 1 mPa-20 kPa, in contact with the gel. In some embodiments, the invention provides a device comprising a gel having an Elastic shear modulus of 0.025-20 kPa, and at least one sensor responsive to mechanical stress signals ranging between 1 mPa-20 kPa, in contact with the gel. In some embodiments, the sensor is responsive a force equivalent to a stress signal ranging between 1 mPa-20 kPa.

The present invention is based, in part, on the finding that different normal, benign and cancer cells have differential indentation activity. Specifically, as exemplified hereinbelow, highly metastatic cells from cell lines and cells from fresh tumors were found to have more indenting cells on the gels compared to benign, non- or low-metastatic cell lines or non-invasive tumors. Furthermore, metastatic cells were shown to have a significantly increased indentation activity compared to non-metastatic cancer cells.

In some embodiments, devices and methods of the invention are directed to diagnosis and prognosis of cancer in a subject. In some embodiments, cells having higher indentation activity correlate with a reduced subject's wellbeing. In some embodiments, reduced wellbeing comprises the existence of a cancerous cell, a cancerous tumor, cancer, or any combination thereof. In some embodiments, reduced wellbeing comprises formation of metastases. In some embodiments, a subject having cells with higher indentation activity has a poor prognosis. In some embodiments, a subject having cells with higher indentation activity has a high likelihood for local invasiveness or distant metastases.

### Indentation Activity and Cells

In some embodiments, devices and methods of the present invention are directed to determine indentation activity of a cell. As defined herein, the term activity” or “indentation parameter” refers to any process in which a cell attempts to penetrate, indents or penetrates a surface. In some embodiments, a cell indents a surface by applying a physical force against the surface. In some embodiments, a cell performing indentation activity attains a morphology or shape, including, but not limited to, spheroidal, rounded, mushroom-like, blebbing or skirt-like morphology. The terms “indentation activity” and “indentation capacity” are used herein interchangeably.

In some embodiments, indentation of a surface encompasses one or more of activities selected from pressuring, compressing, straining, penetrating, squeezing, pushing, shearing, moving, eroding, or degrading the surface. In some embodiments, a cell performing indentation activity indicates the cell has high probability of infiltrating a target. In some embodiments, a target is a tissue. In some embodiments, a target is interstices. Non-limiting examples of a target include, but are not limited to, fat tissue, muscle tissue, blood vessel lining, between cells of a similar type (whether normal or malignant, or others). In some embodiments, the extent of indentation activity varies among cells of different types or origin. In one embodiment, an infiltrating cell has an increased indentation activity. In some embodiments, increased indentation activity is determined relatively to a cell of a non- or a low indentation activity. In some embodiments, a cell obtained or derived from a tumor has increased indentation activity compared to a cell obtained from a non-tumor site of same tissue, organ, or organism.

In some embodiments, increased indentation activity comprises increased indentation frequency (e.g., referring to more indentation attempts per a defined time period). In one embodiment, a cell having increased indentation activity applies more force, mechanical stress or pressure against the surface compared to a cell of a non- or a low-indentation activity. In one embodiment, a cell having increased indentation activity indicates the cell has high probability of penetrating deeper into the surface compared to a cell of a non- or a low indentation activity. In one embodiment, a cell having increased indentation activity indicates the cell has high probability of penetrating faster into the surface compared to a cell of a non- or a low indentation activity. In one embodiment, a cell having increased indentation activity indicates the cell has high probability of having a prolonged penetration durability, i.e., penetration attempts occurring continuously or intermittently over a longer period of time, compared to a cell of a non- or a low indentation activity.

As defined herein, the term “baseline level” and “control” are interchangeable and refer to a cell indentation activity measured in the subject before or at early tumorigenesis. In one embodiment, before is at least 1 week, at least 1 month, at least 3 months, at least 6 months, at least 9 months or at least 12 months before, or at early tumorigenesis. Each possibility represents a separate embodiment of the invention. In another embodiment, a control comprises a non-afflicted cell or tissue obtained from the same subject, such as an adjacent, non-cancerous tissue in the same organ. In one embodiment, a control comprises a non-afflicted control subject. In some embodiments, a control comprises a cell line. In some embodiments, a control comprises the same cell or the same cell population having its indentation activity measured on a gel having different Young's modulus.

In some embodiments, increased indentation activity is at least 5%, at least 10%, at least 35%, at least 50%, at least 100%, at least 250%, at least 500%, or at least 1,000% greater compared to control, or any value and range therebetween. Each possibility represents a separate embodiment of the invention. In some embodiments, increased indentation activity is 5-30%, 25-75%, 50-200%, 100-350%, 250-550%, 500-750%, or 750-1,000% greater compared to control. Each possibility represents a separate embodiment of the invention. In some embodiments, increased indentation activity is by at least 2-fold, at least 5-fold, at least 8-fold, at least 10-fold, at least 20-fold, at least 30-fold, at least 50-fold, at least 75-fold, or at least 100-fold greater compared to control, or any value and range therebetween. Each possibility represents a separate embodiment of the invention.

In some embodiments, an infiltrating cell is a cancer cell. In some embodiments, a cancer cell is a malignant cancer cell. In some embodiments, a malignant cancer cell is a metastatic cancer cell. In some embodiments, a metastatic cancer cell is a high metastatic potential (MP) cancer cell or low MP cancer cell. In some embodiments, the descending order of cells based on their indentation activity from high to low is high MP cancer cell, low MP cancer cell, locally invasive cancer cell, non-metastatic cancer cell, pre-cancerous cell, and non-cancerous cell, including but not limited to a benign cell or a normal cell. In some embodiments, the ascending order of cells based on their indentation activity from low to high is non-cancerous cell, non-metastatic cancer cell, pre-cancerous cell, locally invasive cancer cell, low MP cancer cell and high MP cancer cell.

In some embodiments, an infiltrating cell is an immune cell. The term “immune cell” refers to any cell of the immune system taking part in defending an organism's body, such as from a parasite. Types of immune cells and methods of isolation thereof would be apparent to one of ordinary skill in the art.

According to some embodiments, methods of the invention are directed to determine a type or a stage of a metastatic, cancerous, pre-cancerous or benign tumor based on determining the cell indentation activity. In some embodiments, cell indentation activity of a tumor is indicative of the tumor's specific stage. In some embodiments, the tumor stage includes or correlates with high likelihood for metastasis formation. In some embodiment, staging a tumor is utilized for personalized medical treatment of a subject afflicted with cancer. In some embodiments, a metastatic tumor has a high level of indenting cells. In some embodiment, the personalized medical treatment of a subject afflicted with cancer comprises a step of reducing cell indentation activity.

In some embodiments, the device and method of the invention may be used for determining or characterizing a tumor tissue as a highly metastatic tumor, a low-metastatic potential tumor, a malignant non-metastatic tumor, a pre-malignant tumor with or without lesion and a benign tumor based on the tissue's indenting cell content. In some embodiments, a metastatic tumor comprises at least 10%, at least 20%, at least 30%, at least 40%, at least 50%, at least 60%, at least 70%, at least 80%, or at least 90% indenting cells, or any value and range therebetween. Each possibility represents a separate embodiment of the invention. In some embodiments, a metastatic tumor comprises 5-15%, 10-25%, 20-35%, 27-45%, 40-60%, 55-75%, 70-90%, or 85-100% indenting cells. Each possibility represents a separate embodiment of the invention. In some embodiments, a metastatic tumor or a locally invasive tumor comprises at least 10%, at least 20%, at least 30%, at least 40%, at least 50%, at least 60%, at least 70%, at least 80%, at least 90%, or at least 100% more indenting cells compared to malignant non-metastatic tumor, benign tumor, or normal cells, or any value and range therebetween. Each possibility represents a separate embodiment of the invention. In some embodiments, a metastatic tumor comprises 5-10%, 8-20%, 15-30%, 25-40%, 35-50%, 45-60%, 55-70%, 65-80%, 85-90%, 90-100%, 95-120%, 150-200%, 175-500%, 500-2,000%, 1,500-4,000%, or 2,500-5,000% more indenting cells compared to malignant non-metastatic tumor, benign tumor, or normal cells. Each possibility represents a separate embodiment of the invention. In some embodiments, a metastatic tumor comprises at least 2-fold, at least 5-fold, at least 10-fold, at least 15-fold, at least 25-fold, at least 30-fold, at least 35-fold, at least 40-fold, at least 50-fold, at least 70-fold, at least 85-fold, or at least 100-fold more indenting cells compared to malignant non-metastatic tumor, pre-malignant tumors or lesions or benign tumor, or any value and range therebetween. Each possibility represents a separate embodiment of the invention.

According to some embodiments, the invention is directed to methods of determining indentation activity of a cell. In some embodiments, indentation activity is determined for an infiltrating cell. As used herein, the term “cell infiltration” encompasses migration, transmigration, dissemination, spreading, intravasation, extravasation, invasion, metastasis or any synonym thereof, describing a cell translocating from its source of origin, into other adjacent, nearby or distant organs or tissues. In some embodiments, cell infiltration encompasses cell migration including, but not limited to, any process involving the transition of a cell between different sites. In some embodiments, cell migration is characterized by any one of the sub-processes selected from polarization, protrusion, adhesion, detachment, or cell body translocation. In one embodiment, cell migration is homing.

In some embodiments, the invention is directed to determining indentation activity of a proliferating cell. In some embodiments, cell proliferation encompasses any condition in which cell division rate is greater than the rates of cell death or differentiation. In some embodiments, a proliferating cell comprises a regulated proliferating cell, including, but not limited to, a lymphocyte. In some embodiments, a proliferating cell comprises a dysregulated or an unregulated proliferating cell including, but not limited to, a cancer cell. Non-limiting examples of a cancer cell include a malignant cancer cell, a metastatic cancer cell, a carcinoma cell, an adenoma cell, a lymphoma cell, or others.

### Gels

In some embodiments, the invention provides a device comprising a gel having a stiffness of 0.1-20 kPa. As used herein, the term “gel” refers to any three-dimensional cross-linked network within a liquid. Three-dimensional shapes may include, but are not limited to: filaments, networks, films, ribbons, cords, sheets, flat discs, cylinders, spheres, 3-dimensional amorphous shapes, etc.

In another embodiment, a gel is a dispersion of molecules of a liquid within a solid. In one embodiment, the liquid particles are dispersed in the solid medium. In some embodiments, the liquid is water or a water-based liquid. In one embodiment, a water-based liquid comprises cell culture media. In some embodiments, the gel of the invention comprises at least 5%, at least 10%, at least 20%, at least 35%, at least 50%, at least 60%, at least 75%, at least 85%, at least 90%, at least 95%, or at least 99% water, or any value and range therebetween. Each possibility represents a separate embodiment of the invention. In some embodiments, the gel of the invention comprises 1-5%, 4-10%, 8-20%, 15-35%, 30-50%, 40-60%, 55-75%, 70-85%, 80-90%, 85-95%, or 90-99% water. Each possibility represents a separate embodiment of the present invention.

In some embodiments, the gel comprises a polymer that provides or comprises a surface, a layer, or a coating suitable for adherence/attachment, infiltration, penetration and indentation of cells. In some embodiments, the polymer is biocompatible. As used herein, “biocompatible” means the ability of an object to be accepted by and to function in a recipient without eliciting a significant foreign body response (such as, for example, an immune, inflammatory, thrombogenic, or the like response) and without having direct cell toxicity, or cytotoxicity. In some embodiments, the polymer is biologically inert. As used herein, “biologically inert” refers to a material which does not initiate a response or interact when introduced to a biological cell or tissue.

In one embodiment, a gel has at least 5%, at least 10%, at least 20%, at least 30%, at least 40%, at least 50%, at least 60%, at least 70%, at least 75%, at least 80%, at least 85%, at least 90%, at least 92%, or at least 95% porosity, or any value and range therebetween. Each possibility represents a separate embodiment of the invention. In another embodiment, a gel has 5-15%, 10-25%, 20-45%, 40-50%, 45-60%, 55-70%, 65-75%, 70-80%, 60-85%, 75-90%, 77-92%, or 85-95% porosity. Each possibility represents a separate embodiment of the present invention.

In another embodiment, a gel has a pore average diameter of 1 nm at most, 5 nm at most, 10 nm at most, 20 nm at most, 30 nm at most, 40 nm at most, 50 nm at most, 60 nm at most, 70 nm at most, 80 nm at most, 90 nm at most, 100 nm at most, 150 nm at most, 200 nm at most, 250 nm at most, 300 nm at most, 350 nm at most, 400 nm at most, 425 nm at most, 450 nm at most, 475 nm at most, 500 nm at most, 750 nm at most, 1 μm at most, 2 μm at most, or 3 μm at most, or any value and range therebetween. Each possibility represents a separate embodiment of the invention. In another embodiment, a gel has a pore average diameter of 1-5 nm, 4-10 nm, 8-20 nm, 15-30 nm, 25-40 nm, 35-50 nm, 40-60 nm, 50-70 nm, 65-80 nm, 75-90 nm, 85-100 nm, 90-110 nm, 105-120 nm, 100-130 nm, 115-140 nm, 120-150 nm, 135-160 nm, 140-170 nm, 150-180 nm, 165-190 nm, 175-200 nm, 190-250 nm, 220-300 nm, 275-400 nm, 350-425 nm, 415-500 nm, 450-850 nm, 800-1,200 nm, or 1-3 μm. Each possibility represents a separate embodiment of the present invention.

The porosity of the gel may be controlled by a variety of techniques known to those skilled in the art. In another embodiment, as the porosity is increased, use of polymers having a higher shear or Young's modulus, addition of stiffer polymers as a co-polymer or mixture, addition of combinations of monomers or cross-linkers adding stiffness, or an increase in the cross-link density of the polymer are used to increase the stability of the gel with respect to cellular invasion or indentation.

In some embodiments of the methods of the invention, a cell is seeded on a gel as defined herein. In one embodiment, a cell is allowed to settle on the gel. In one embodiment, the cell is allowed to settle on the gel prior to monitoring, such as monitoring of cell indentation. In some embodiments, the terms “settle”, “contact”, “attach” and “adhere” are used herein interchangeably. In one embodiment, a cell is allowed to settle on the gel for at least 1 min, at least 2 min, at least 5 min, at least 10 min, at least 15 min, at least 20 min, at least 30 min, at least 40 min, at least 50 min, at least 60 min, at least 70 min, at least 80 min, at least 90 min, at least 100 min, at least 110 min, or at least 120 min, or any value and range therebetween. Each possibility represents a separate embodiment of the invention. In one embodiment, a cell is allowed to settle on the gel for 0.5-1 min, 1-2 min, 1.5-5 min, 3-10 min, 7-15 min, 10-20 min, 12-30 min, 20-40 min, 35-50 min, 40-60 min, 70-120 min, 100-240 min, 220-360 min or 300-480 min. Each possibility represents a separate embodiment of the present invention.

In another embodiment, a cell is allowed to settle on the gel of the invention for at least 5 min in-vitro or ex-vivo, in order to reach baseline indentation rates.

In some embodiments, the indentation activity of a cell is determined at least 5 min, at least 15 min, at least 30 min, at least 60 min, at least 1 hr, at least 2 hr, at least 3 hr, at least 4 hr, at least 6 hr, at least 8 hr, at least 10 hr, at least 12 hr, at least 16 hr, at least 20 hr, or at least 24 hr, or any value and range therebetween, after the period of cell settling or adherence was completed. Each possibility represents a separate embodiment of the invention. In some embodiments, the indentation activity of a cell is determined 5-25 min, 15-45 min, 30-70 min, 1-3 hr, 2-5 hr, 3-6 hr, 4-8 hr, 6-9 hr, 7-11 hr, 10-14 hr, 13-18 hr, 16-20 hr, or 18-24 hr, after the period of cell settling or adherence was completed. Each possibility represents a separate embodiment of the invention.

In another embodiment, a gel such as described herein is 30-50 μm thick. In another embodiment, the gel is 40-60 μm thick. In another embodiment, the gel is 50-70 μm thick. In another embodiment, the gel is 60-90 μm thick. In another embodiment, the gel is 80-110 μm thick. In another embodiment, the gel is 85-120 μm thick. In another embodiment, the gel is 90-150 μm thick. In another embodiment, the gel is 115-145 μm thick. In another embodiment, the gel is 130-175 μm thick. In another embodiment, the gel is 150-190 μm thick. In another embodiment, the gel is 170-220 μm thick. In another embodiment, the gel is 180-235 μm thick. In another embodiment, the gel is 190-240 μm thick. In another embodiment, the gel such is 200-250 μm thick. In another embodiment, the gel such is 250-280 μm thick.

In another embodiment, the gel comprises a material selected from: polyacrylamide (PAM), collagen-GAG, collagen, fibrin, fibronectin, poly-1-lactic acid (PLLA), polylactic glycolic acid (PLGA) PLLA-PLGA co-polymer, poly(anhydride), poly(hydroxy acid), poly(ortho ester), poly(propylfumerate), poly(caprolactone), polyamide, polyamino acid, polyacetal, polycyanoacrylate, polyurethane and polysaccharide, polypyrrole, polyaniline, polythiophene, polystyrene, polyester, polyurea, poly(ethylene vinyl acetate), polypropylene, polymethacrylate, polyethylene, polycarbonate, poly(ethylene oxide), polypyrrole, polycaprolactone and poly(ethersulfone), poly(acrylonitrile-co-methylacrylate) (PAN-MA), or silicone. Each possibility represents a separate embodiment of the invention.

In some embodiments, the gel of the invention comprises particles. In some embodiments, the particles are nanoparticles. In some embodiments, the particles are fluorescent particles. In some embodiments, fluorescent nanoparticles are observed according to any method known in the art, such as, but not limited to, excitation, emission and detection using a fluorescent microscope, for example, a confocal microscope. In some embodiments, fluorescent nanoparticles are used as location markers. In some embodiments, the particles are localized at the gel surface. In some embodiments, the particles are immobilized at the gel surface. The terms “particles” and “nanoparticles” are used herein interchangeably.

In some embodiments, the particles are at least 5 nm, at least 10 nm, at least 50 nm, at least 75 nm, at least 100 nm, at least 150 nm, at least 200 nm, at least 350 nm, at least 425 nm, or at least 500 nm in diameter, or any value and range therebetween. Each possibility represents a separate embodiment of the invention. In some embodiments, the particles are 5-50 nm, 25-75 nm, 70-150 nm, 100-200 nm, 175-300 nm, 225-400 nm, or 350-500 nm in diameter. Each possibility represents a separate embodiment of the invention.

In another embodiment, the gel further comprises a cell adhesion promoting agent, a proliferation inducer, a differentiation inducer, an extravasation inducer, a migration inducer, a senescence inducer, a cell-death promoting compound, or any combination thereof. Each possibility represents a separate embodiment of the invention. In some embodiments, the cell is contacted with a gel, wherein the gel is incubated in a solution comprising a cell adhesion promoting agent, a proliferation inducer, a differentiation inducer, an extravasation inducer, a migration inducer, a senescence inducer, a cell-death promoting compound, or any combination thereof. Each possibility represents a separate embodiment of the invention. In another embodiment, the gel further comprises a cell adhesion protein, a growth factor, a cytokine, a hormone, a protease, a protease substrate, or any combination thereof. Each possibility represents a separate embodiment of the invention. In another embodiment, any substance as described herein is attached to the gel. In another embodiment, any substance as described herein is embedded within the gel. In another embodiment, any substance as described herein is impregnated within the gel.

In some embodiments, the method of the invention comprises supplementing a gel of the invention, or a solution in which the gel is incubated, with a cell adhesion promoting agent, a proliferation inducer, a differentiation inducer, an extravasation inducer, a migration inducer, a senescence inducer, a cell-death promoting compound, a cell adhesion protein, a growth factor, a cytokine, a hormone, a protease, a protease substrate, or any combination thereof.

The stiffness of the gel may be controlled by a variety of techniques known to those skilled in the art. As defined herein, the term “stiffness” refers to a shear modulus. In some embodiments, the term “shear modulus” refers to an “elastic modulus”. In some embodiments, elastic modulus refers to Young's modulus. In some embodiments, the term elastic modulus is determined by response of a material to application of tensile stress or strain. In some embodiments, the term “elastic modulus” is determined by response of a material to application of shear stress or strain (e.g., according to any procedure known in the art).

In some embodiments, the gel of the invention has a stiffness of at least 0.1 kPa, at least 0.5 kPa, at least 1 kPa, at least 2 kPa, at least 5 kPa, at least 10 kPa, at least 12 kPa, at least 15 kPa, at least 20 kPa, at least 25 kPa, at least 30 kPa, at least 35 kPa, at least 40 kPa, at least 45 kPa, at least 50 kPa, at least 60 kPa, at least 70 kPa, at least 80 kPa, at least 85 kPa, at least 90 kPa, or at least 100 kPa, or any value and range therebetween. In some embodiments, the gel of the invention has a stiffness of 0.1-0.2 kPa, 0.15-0.5 kPa, 0.4-1 kPa, 0.75-2 kPa, 1.5-5 kPa, 4-10 kPa, 8-12 kPa, 11-15 kPa, 14-20 kPa, 17-25 kPa, 20-30 kPa, 25-35 kPa, 30-40 kPa, 37-45 kPa, 40-50 kPa, 48-60 kPa, 55-70 kPa, 65-80 kPa, 75-85 kPa, 70-90 kPa, or 88-100 kPa. Each possibility represents a separate embodiment of the present invention. In some embodiments, the stiffness of the gel of the invention is determined by a rheometer, such as exemplified herein.

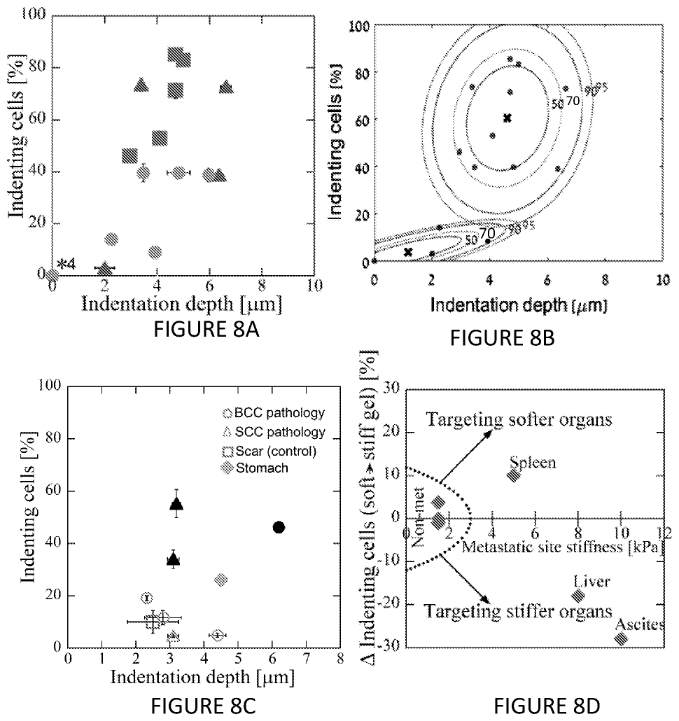

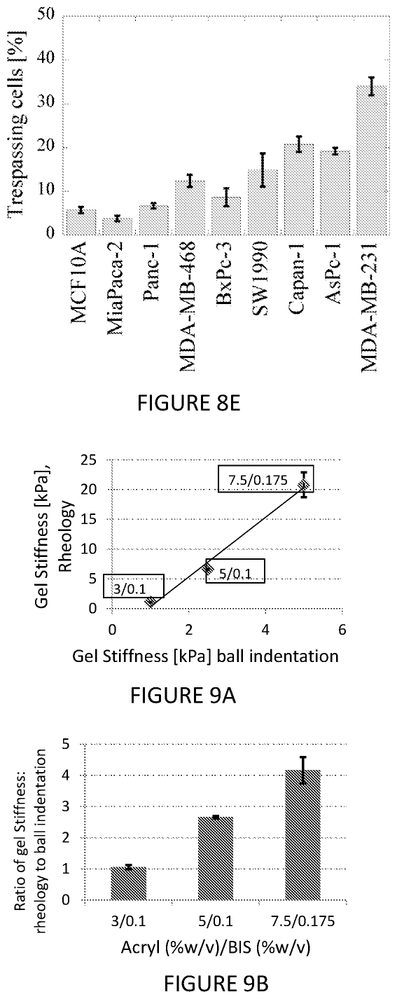

The choice of polymer and the ratio of polymers in a co-polymer of a gel, or the choice of monomers and cross-linker and their ratio within the gel of the invention may be adjusted to optimize the stiffness and porosity of the gel or either of the parameters. In another embodiment, the molecular weight and cross-link density of the gel is regulated to control both the mechanical properties of the gel and the indentation rate. In another embodiment, the mechanical properties are optimized to mimic those of the tissue of origin or an expected invasion site (such as exemplified in FIG. 8D). In another embodiment, materials of the gel comprise natural or synthetic organic polymers that can be gelled, or polymerized or solidified (e.g., by aggregation, coagulation, hydrophobic interactions, or cross-linking) into a hydrogel e.g., structure that entraps, encloses water and/or other molecules, which allows exchange of molecules between the gel and the gel's outer surroundings.

In another embodiment, polymers used in the gel are biocompatible, biodegradable, and/or bioerodible and act as adhesive substrates for cells. In another embodiment, the polymers of the outer layer, or the layer that is directly contacting the outer surroundings of the gel, or the layer that is directly in contact with external elements (e.g. cells), comprise non-resorbing or non-biodegradable polymers or biologically inert or bioinert materials. The phrase “non-biodegradable polymer”, as used herein, refers to any polymer or polymers which at least substantially (i.e. more than 50%) do not degrade or erode in vitro, ex vivo or in-vivo. The terms “non-biodegradable”, “non-resorbing” are equivalent and are used interchangeably herein. The terms “biologically inert” or “bioinert” are equivalent and are used interchangeably herein.

In another embodiment, the gel comprises polymers, such as, fibrinogen, fibrin, thrombin, chitosan, collagen, alginate, poly(N-isopropylacrylamide), hyaluronate, albumin, synthetic polyamino acids, prolamines, acrylamide, Bis-acrylamide, polyacrylamide, polysaccharides such as alginate, heparin, and other naturally occurring biodegradable polymers of sugar units. In another embodiment, the gel comprises materials which are ionic hydrogels, for example, ionic polysaccharides, such as alginates or chitosan. Ionic hydrogels may be produced by cross-linking the anionic salt of alginic acid, a carbohydrate polymer isolated from seaweed, with ions, such as calcium cations. In some embodiments, the gel comprises synthetic polymers, such as polysiloxanes (i.e., silicone) comprising polydimethylsiloxane, methyl trichlorosilane and methyl trimethoxysilane. In another embodiment, the gel of the invention comprises any one of the aforementioned polymers in at least one of the gel's layers.

In another embodiment, the gel of the invention is made by any of a variety of techniques known to those skilled in the art. Salt-leaching, porogens, solid-liquid phase separation (sometimes termed freeze-drying), spin coating, and phase inversion fabrication are used, in some embodiments, to produce gels. A non-limiting example for preparing a gel of the invention includes, but not limiting to, mixing the monomer with a cross-linker in the presence of an initiator and catalyst, as would be apparent to any one of ordinary skill in the art.

### Sensors

In some embodiments, the device of the invention comprises a sensor. In some embodiments, the device comprises at least one sensor. In some embodiments, the sensor is in contact with the gel of the invention. In some embodiments, the sensor can be located operatively on top, below, around and within the gel. In some embodiments, the sensor is a force sensor. In some embodiments, the sensor measures force inputs, and converts those inputs to stress or pressure outputs. In some embodiments, the sensor is a displacement sensor. Non-limiting example of a displacement sensor includes, but is not limited to, laser displacement sensors. In some embodiments, the sensor is an optic sensor. In some embodiments, the optic sensor is located on top of the gel, below the gel, at a side of the gel, or a combination thereof. In some embodiments, the optic sensor is a microscope, or a camera, such as a digital camera, or any other detecting apparatus capable of detecting objects in a wide range of wavelengths, including but not limited to the visible light wavelength, objects emitting fluorescence, or others.

In one embodiment, the sensor is a pressure sensor. Types of pressure sensors are well known to one of ordinary skill in the art. Non-limiting examples of pressure sensors include sensors based on thermal micro-flow measurement, capacitive microelectromechanical systems (MEMS) sensor, vacuum pressure sensor, lateral nano-Newton force piezoresistive sensor, and others. In one embodiment, lateral nano-Newton force piezoresistive sensor can measure forces as low as 5 nN. In one embodiments, capacitive CMOS-MEMS force sensor can measure forces as low as 2 pN.

In some embodiments, a sensor-based system can be improved by using piezochromatic materials as the pressure sensors. As defined herein, a “piezochromatic material” refers to any material exhibiting a pressure-dependent reversible shift of the selective reflection wavelength (i.e., mechanochromic activity). A non-limiting example includes, but not limited to, a mechanochromic photonic gel based on colloidal crystalline array that is sensitive to pressure on a single kPa scale, and changes color in the range from red to blue (Δλ=150 nm).

In some embodiments, a sensor of the invention comprises a strain sensor. As used herein, the terms “strain sensor”, “strain transducer” and “strain gauge” are interchangeable. In some embodiments, a strain sensor comprises a quarter-, a half-, or a full-bridge strain sensor. Non-limiting examples of strain sensors include: active strain transducer, piezoelectric strain transducer and optical strain sensor.

In some embodiments, the device of the invention further comprises a second sensor. In some embodiments, the second sensor is a pH sensor or a temperature sensor.

In some embodiments, the sensor senses any cell indentation activity or a related outcome thereof, in “real time”. In some embodiments, an outcome of cell indentation comprises altered acidity, altered temperature, or both. The term “altered” encompasses an increase, or a decrease.

In some embodiments, the device of the invention further comprises a detecting apparatus, including, but not limited to a microscope, or a camera, such as a digital camera, or any other detecting apparatus capable of detecting objects in a wide range of wavelengths, including but not limited to the visible light wavelength, objects emitting fluorescence, or others. In some embodiments, the detecting apparatus is further coupled to a computer program. In some embodiments, the detecting apparatus captures and digitizes an input including, but not limited to, a single cell or a group of cells adhered to a gel surface. In some embodiments, the detecting apparatus captures and digitizes an input prior to analysis, such as by a computer program. A non-limiting example for use of a detecting apparatus includes: seeding an estimated number of cells on a gel of the invention, capturing the cells adhered to the gel's surface using the detecting apparatus which subsequently transfers the captured image to a computer program capable of computing and outputting the exact number of cells adhered to the gel's surface. In some embodiments, a computer program generates an output. In some embodiments, the output comprises the number, percent, or both, of indenting cells out of the total adhered cells. In some embodiments, the output comprises the indentation depth of indenting cells. In some embodiments, the output comprises the number, percent, or both, and the indentation depth of indenting cells.

### Methods of Use

According to some embodiment, there is provided a method of determining indentation activity of a cell population, the method comprising: contacting the cell population with a gel having a Young's modulus of 0.1-20 kPa; and measuring a cell indentation parameter using at least one sensor responsive to signals ranging between 1 mPa-20 kPa, wherein an increase in the cell indentation parameter is indicative of indentation activity of the cell population.

A method of classifying a cell population according to indentation activity, the method comprising: contacting a cell population with a gel having a Young's modulus of 0.1-20 kPa; measuring a cell indentation parameter, thereby determining the cell population indentation activity; and determining a cell characteristic of the cell population based on a pre-determined indentation activity threshold, wherein the cell characteristic is selected from the group consisting of: invasiveness, infiltration, and differentiation state, thereby classifying the cell population according to the indentation activity.

In some embodiments, the cell indentation parameter is selected from: number of indenting cells, indentation depth attained by the cells, force applied by the cells to the gel, pressure applied by the cells to the gel, strain applied by the cells to the gel, displacement applied by cells to the gel, or any combination thereof.

According to some embodiments, there is provided a composition comprising a gel having a Young's modulus of 0.1-20 kPa for use in classifying a cell population according to an indentation activity.

In some embodiments, methods of the present invention are directed to determining or quantifying the indentation activity of any one of a single cell, a cell population, multiple cells, a group of cells, a cluster of cells, an aggregate of cells, or a spheroid of cells. The terms “single cell”, “cell population”, “multiple cells”, “group of cells” “cluster of cells”, “aggregate of cells” and “spheroid of cells” are used herein interchangeably.

In some embodiments, the method is directed to an impenetrable gel having a Young's modulus of 0.1-20 kPa, for use in determining the indentation activity of a cell.

In some embodiments, the cell population is obtained from a sample being obtained from a subject.

In some embodiments, the method comprises a step of quantifying the cell population indentation activity, e.g., the number of indenting cells, the depth attained by the cells, or both, for diagnosing cancer in a subject, wherein increased indentation activity of the cell population relative to control is indicative of cancer in the subject.

In some embodiments, the method further comprises a step of quantifying the cell population indentation activity, wherein increased indentation activity of the cell population relative to control provides a prediction or prognosis of metastatic cancer in the subject.

In some embodiments, the prediction of the metastatic cancer comprises predicting the target organ for metastases by comparing the indentation activity of the cell population on a second gel having a different stiffness, or different Young's modulus compared to a first gel. In one embodiment, the stiffness or the Young's modulus of the first gel, of the second gel, or both, is indicative of the stiffness of a target organ.

In some embodiments, the method comprises comparing the indentation activity on at least two gels having different stiffness values, Young's moduli, different pore size, or any combination thereof. In some embodiments, the difference in the Young's moduli of the gels is at least 50 Pa, at least 100 Pa, at least 250 Pa, at least 350 Pa, at least 400 Pa, at least 500 Pa, at least 1 kPa, at least 2 kPa, at least 3 kPa, at least 5 kPa, at least 8 kPa, at least 10 kPa, at least 12 kPa, at least 15 kPa, or at least 19 kPa, or any value and range therebetween. Each possibility represents a separate embodiment of the invention. In some embodiments, the difference in the Young's moduli of the gels is 50-250 Pa, 100-350 Pa, 150-400 Pa, 200-500 Pa, 300 Pa to 1,200 Pa, 1-3 kPa, 2-5 kPa, 4-9 kPa, 8-13 kPa, 12-16 kPa, or 15-19 kPa. Each possibility represents a separate embodiment of the invention.

In some embodiments, the first gel has a greater Young's modulus compared to the Young's modulus of the second gel. In some embodiments, the first gel has a lower Young's modulus compared to the Young's modulus of the second gel.

In some embodiments, cell indentation activity is measured on a stiff gel and is thereafter measured on a softer gel. In some embodiments, cell indentation activity is measured on a soft gel and is thereafter measured on a stiffer gel.

In some embodiments, the cell indentation activity of portions, representatives, fractions, or any distribution of a cell population which provides sub-samples or sub-populations which are equivalent or essentially the same, is compared on gels having different mechanical properties as disclosed herein.

In some embodiments, cell indentation activity is measured on a gel having a greater Young's modulus compared to the Young's modulus of the second gel, and is thereafter measured on a gel having a lower Young's modulus compared to the Young's modulus of the first gel. In some embodiments, cell indentation activity is measured on a gel having a lower Young's modulus compared to the Young's modulus of the second gel, and is thereafter measured on a gel having a greater Young's modulus compared to the Young's modulus of the first gel.

In some embodiments, indentation activity is measured on a first gel having a low Young's modulus value, for example 1-15 kPa, on a second gel having a higher Young's modulus, for example 2-16 kPa, and then the indentation activity of the two measurements are compared, wherein increased indentation activity or reduced indentation activity is concluded. In some embodiments, increased or reduced indentation activity concluded from different gel stiffnesses (i.e., different Young's modulus) is predictive of site of metastases. In some embodiments, a cell or a cell population having increased indentation activity on a low Young's modulus gel such as 1-5 kPa compared to the indentation activity of the cell or cell population on a higher Young's modulus gel for such as 2-16 kPa is indicative of the cell or cell population is likely to metastasizes a soft tissue. In some embodiments, a cell or a cell population having reduced indentation activity on a low Young's modulus gel such as 1-15 kPa compared to the indentation activity of the cell or cell population on a higher Young's modulus gel for such as 2-16 kPa is indicative of the cell or cell population is likely to metastasizes a stiffer tissue.

In some embodiments, a reduced indentation activity of a cell population determined on a softer gel compared to the cell indentation on a stiffer gel, as exemplified herein (such as in FIG. 8D), is indicative of the tendency of cancerous cells to metastasize a stiffer organ.

As demonstrated herein, such as in the case of pancreatic cancer cells, a difference of as little as 1 kPa in gel stiffness, is sufficient to discriminate metastatic from non-metastatic cells (such as in FIG. 8D), or determine stiffness of target tissue of the metastatic cells (FIG. 8D), or both.

In some embodiments, reduced indentation activity of the cell population on a first gel having a Young's modulus of at least 0.1 kPa, at least 0.5 kPa at least 1 kPa, at least 2 kPa, at least 3 kPa, at least 4 kPa, at least 5 kPa, at least 7 kPa, at least 9 kPa, at least 10 kPa, at least 12 kPa, at least 15 kPa, or at least 18 kPa, or any value and range therebetween, compared to the indentation activity of the cell population on a second gel having a Young's modulus of at least 1 kPa, at least 2 kPa, at least 3 kPa, at least 4 kPa, at least 5 kPa, at least 6 kPa, at least 7 kPa, at least 8 kPa, at least 9 kPa, at least 10 kPa, at least 11 kPa, at least 13 kPa, at least 15 kPa, at least 17 kPa, or at least 19 kPa, or any value and range therebetween, is predictive of the cell population target organ for metastases is an organ being essentially the same or at least 1.5-fold, at least 2-fold, at least 3-fold, at least 5-fold, at least 7-fold, at least 10-fold, at least 20-fold, at least 100-fold, at least 200-fold, at least 500-fold, or at least 1,000-fold stiffer than the gel having the greater stiffness, or any value and range therebetween. Each possibility represents a separate embodiment of the invention. In some embodiments, reduced indentation activity of the cell population on a gel having a Young's modulus of 0.1-3 kPa, 0.5-4 kPa, 1-5 kPa, 2-7 kPa, 3-5 kPa, 1-4 kPa, or 2-5 kPa, compared to the indentation activity of the cell population on a gel having a Young's modulus of 1-4 kPa, 1.5-5 kPa, 2-6 kPa, 3-6.5 kPa, 4-7 kPa, 6-10 kPa, 7-11 kPa, 5-8 kPa, 4-9 kPa, 7-10 kPa, 6-11 kPa, 8-13 kPa, or 10-20 kPa, is predictive of the cell population target organ for metastases is an organ being 1-5-fold, 3-9-fold, 8-15-fold, 10-100-fold, 20-250-fold, 200-500-fold, or 400-1,000-fold stiffer than the gel having the greater stiffness. Each possibility represents a separate embodiment of the invention.

In some embodiments, increased indentation activity of the cell population on a first gel having a Young's modulus of at least 0.1 kPa, at last 0.5 kPa, at least 1 kPa, at least 2 kPa, at least 3 kPa, at least 4 kPa, at least 5 kPa, at least 6 kPa, at least 7 kPa, at least 8 kPa, at least 9 kPa, at least 10 kPa, at least 11 kPa, at least 13 kPa, at least 15 kPa, at least 17 kPa, or at least 19 kPa, or any value and range therebetween, compared to the indentation activity of the cell population on a second gel having a greater Young's modulus of 1 kPa, at least 2 kPa, at least 3 kPa, at least 4 kPa, at least 5 kPa, at least 6 kPa, at least 7 kPa, at least 8 kPa, at least 9 kPa, at least 10 kPa, at least 11 kPa, at least 13 kPa, at least 15 kPa, at least 17 kPa, or at least 19 kPa, or any value and range therebetween, is predictive of the cell population target organ for metastases is an organ being essentially the same or at least 2-fold, at least 3-fold, at least 5-fold, at least 7-fold, at least 10-fold, at least 20-fold, at least 100-fold, at least 200-fold, at least 500-fold, or at least 1000-fold softer than the gel having the greater stiffness, or any value and range therebetween. Each possibility represents a separate embodiment of the invention. In some embodiments, increased indentation activity of the cell population on a gel having a Young's modulus of 0.1-3 kPa, 0.5-4 kPa, 1-5 kPa, 2-7 kPa, 3-5 kPa, 1-4 kPa, or 2-5 kPa, compared to the indentation activity of the cell population on a gel having a Young's modulus of 1-4 kPa, 1.5-5 kPa, 2-6 kPa, 3-6.5 kPa, 4-7 kPa, 6-10 kPa, 7-11 kPa, 5-8 kPa, 4-9 kPa, 7-10 kPa, 6-11 kPa, 8-13 kPa, or 10-20 kPa, is predictive of the cell population target organ for metastases is an organ being 1-5-fold, 3-9-fold, 8-15-fold, 10-100-fold, 20-250-fold, 200-500-fold, or 400-1,000-fold softer than the gel having the lower stiffness. Each possibility represents a separate embodiment of the invention.

In some embodiments, cell indentation activity of an infiltrating cell on the described gel with a defined stiffness correlates with infiltration into a target tissue. In some embodiments, the target tissue is a metastatic site tissue. In some embodiments, the target tissue is a non-tumor or tumor adjacent region in a primary site tissue. In some embodiments, an infiltrating cell has a lower indentation activity when infiltrating a softer target tissue compared to when infiltrating a harder target tissue. As defined herein, the terms “soft” or “softer”, and “stiff” or “stiffer” may be relative to one another and refer to a spectrum of Young's moduli of tissues ranging from 10 Pa to 500 kPa, respectively. In some embodiments, a mucosal tissue has a stiffness of 10 Pa. In some embodiments, a brain tissue has a stiffness of 100 Pa. In some embodiments, a lung tissue has a stiffness of about 1,000 Pa. In some embodiments, a liver tissue has a stiffness of about 1,000 Pa. In one embodiment, a liver tissue is stiffer than a lung tissue. In some embodiments, a muscle tissue has a stiffness of about 10,000 Pa or 10 kPa. In some embodiments, any given tissue being malignant cancerous or metastatic is stiffer, or having a greater Young's modulus compared to the same tissue being benign, normal, or non-cancerous. In some embodiments, any given tissue being benign, normal, or non-cancerous is softer, or having a lower Young's modulus compared to the same tissue being malignant, cancerous or metastatic. In some embodiments, a diseased tissue has a greater Young's modulus compared to the same tissue being “normal” or “non-diseased”. Non-limiting examples of soft- or medium-range stiffness organs/tissues including their respective stiffnesses in normal or diseased condition are summarized in the following table:

| | | Stiffness [kPa] | | |
|---|---|---|---|---|
| Organ/tissue | | Normal | Disease | References |
| Soft | Brain | 0.1-0.5 | 2 | Flanagan et al., (2002); |
| | | | | Sakai et al., (2016) |
| | Breast (fat) | 1-3 | 4-5 | Butcher et al., (2009); |
| | | | | Baker et al., (2009) |
| | Liver | 1-2 | 8 | Kohlhass et al., (2012) |
| | Pancreas | 1 | 2-4 | Rice et al., (2017) |
| | Lung parenchyma | 2 | 4 | Engler et al., (2006) |
| | Spleen | 3 | 5 | Ma et al., (2016) |
| | Kidney | 2.4 | 7.4 | Lekka (2016) |
| Medium | Muscular tissues | 10 | 15 | Engler et al., (2006) |
| | Intra-abdominal | 10 | 10 | Stokes and Gardner |
| | ascites | | | (2010) |
| | Bladder | 14 | 28 | Engler et al., (2006) |

In some embodiments, the method is for screening for a compound suitable for reducing indentation activity of the cell population, the method comprising contacting the cell population with the compound, wherein reduction of indentation activity of the cell population in the presence of the compound compared to the indentation activity of the cell population in the absence of the compound indicates the compound is suitable for reducing indentation activity of the cell population.

In some embodiments, the cell population is contacted with the compound prior to contact with the gel. In some embodiment, the cell population is contacted with the compound after contact with the gel. In some embodiment, the cell population is contacted with the compound prior to and after contact with the gel. In some embodiments, contact with the compound comprises incubation with the compound. In some embodiments, incubation is for a period of at least 1 min, at least 5 min, at least 10 min, at least 30 min, at least 60 min, at least 1 hr, at least 2 hr, at least 4 hr, at least 6 hr, at least 12 hr, at least 16 hr, at least 24 hr, or any value and range therebetween. Each possibility represents a separate embodiment of the invention. In some embodiments, incubation is for a period of 1-20 min, 10-45 min, 30-60 min, 1-3 hr, 2-5 hr, 4-8 hr, 6-12 hr, 10-18 hr, 16-24 hr. Each possibility represents a separate embodiment of the invention.

In some embodiments, a compound suitable for reducing indentation activity of a cell is suitable for preventing or reducing cancer invasiveness, progression, metastases, and the like.

In some embodiments, a cell having increased indentation activity applies a force to the gel with a magnitude eliciting a sensor's measuring output of 0.1-5 N. In some embodiments, the cell applies the force to the gel surface. In some embodiments, the sensor measures the applied force at the gel surface, within the gel, under the gel, around the gel, in a single location in the gel, in multiple locations in the gel, in a single layer of the gel, in multiple layers of the gel, or any combinations thereof. In some embodiments, the measured force applied by a cell having increased indentation activity is of at least 1 nN, at least 5 nN, at least 10 nN, at least 20 nN, at 30 nN, at least 40 nN, at least 50 nN, at least 60 nN, at least 70 nN, at least 80 nN, at least 90 nN, at least 100 nN, at least 200 nN, at least 300 nN, at least 400 nN, at least 500 nN, at least 750 nN, at least 900 nN, at least 1 N, at least 2 N, at least 3 N, at least 4 N, at least 5 N, at least 6 N, at least 7 N, at least 8 N, at least 9 N, or at least 10 N, or any value and range therebetween. Each possibility represents a separate embodiment of the invention. In some embodiments, the measured force applied by a cell having increased indentation activity is of 0.1-1 nN, 0.5-2 nN, 1.5-5 nN, 4-10 nN, 8-20 nN, 15-30 nN, 20-40 nN, 30-50 nN, 40-60 nN, 50-70 nN, 65-80 nN, 75-90 nN, 85-100 nN, 90-150 nN, 100-300 nN, 250-500 nN, 400-750 nN, 700-950 nN, 900-1,500 nN, 1-1.75 N, 1.5-3 N, 2.5-4 N, 3-5.5 N, 5-7.5 N, 7-9 N, or 8-10 N. Each possibility represents a separate embodiment of the invention. The aforementioned values are expected to increase proportionally to the size or number of cells examined according to the disclosed method.

In some embodiments, a cell having increased indentation activity applies pressure to the gel with a magnitude eliciting a sensor's measuring output of 0.001-5,000 Pa, or 1 mPa-5 kPa. In some embodiments, the cell applies the pressure to the gel surface. In some embodiments, the sensor measures the applied pressure at the gel surface, within the gel, under the gel, around the gel, in a single location in the gel, in multiple locations in the gel, in a single layer of the gel, in multiple layers of the gel, or any combinations thereof. In some embodiments, the measured pressure applied by a cell having increased indentation activity is of at least 0.0005 Pa, at least 0.001 Pa, at least 0.01 Pa, at least 1 Pa, at least 10 Pa, at least 50 Pa, at least 100 Pa, at least 250 Pa, at least 400 Pa, at least 600 Pa, at least 750 Pa, at least 1,000 Pa, at least 1,500 Pa, at least 2,500 Pa, at least 3,000 Pa, at least 4,500 Pa, or at least 5,000 Pa, or any value and range therebetween. Each possibility represents a separate embodiment of the invention. In some embodiments, the measured force applied by a cell having increased indentation activity is of 0.001-0.02 Pa, 0.01-0.2 Pa, 0.1-2 Pa, 1-20 Pa, 10-200 Pa, 100-750 Pa, 500-1,250 Pa, 1,000-2,500 Pa, 2,000-3,500 Pa, 3,000-4,500 Pa, or 4,000-6,000 Pa. Each possibility represents a separate embodiment of the invention.

In some embodiments, indentation activity of a cell seeded on a gel of the invention is represented by the gel's displacement. In some embodiments, a gel's displacement is the gel's surface vertical displacement. In one embodiment, a displacement of the gel is indicated by a fiducial marker. In some embodiments, indentation activity of a cell seeded on a gel of the invention is represented by strain magnitude. In some embodiments, the magnitude of strain measured following seeding of a cell having increased indentation activity is at least 0.1 μm, at least 0.2 μm, at least 0.6 μm, at least 1.2 μm, at least 2.4 μm, at least 4.8 μm, at least 6 μm, at least 8 μm, at least 10 μm, at least 12 μm, at least 15 μm, at least 18 μm, or at least 25 μm, or any value and range therebetween. Each possibility represents a separate embodiment of the invention. In some embodiments, the magnitude of strain measured following seeding of a cell having increased indentation activity is 0-0.2 μm, 0.1-0.6 μm, 0.5-1.5 μm, 1-2.5 μm, 2-4.5 μm, 4-7.5 μm, 5-9.5 μm, 8-12 μm, 10-14 μm, 12-15 μm, 14-17 μm, 16-18 μm, 17-20 μm, or 19-25 μm. Each possibility represents a separate embodiment of the invention.

In some embodiments, a cell having increased indentation activity is a cell attempting to penetrate a gel of the invention at least once a minute, at least once every 2 minutes, at least once every 3 minutes, at least once every 4 minutes, at least once every 5 minutes, at least once every 6 minutes, at least once every 7 minutes, at least once every 8 minutes, at least once every 9 minutes, at least once every 10 minutes, at least once every 11 minutes, at least once every 12 minutes, at least once every 13 minutes, at least once every 14 minutes, at least once every 15 minutes, at least once every 16 minutes, at least once every 17 minutes, at least once every 18 minutes, at least once every 19 minutes, at least once every 20 minutes, at least once every 25 minutes, at least once every 30 minutes, at least once every 35 minutes, at least once every 40 minutes, at least once every 45 minutes, at least once every 50 minutes, at least once every 55 minutes, or at least once every 60 minutes, or any value and range therebetween. Each possibility represents a separate embodiment of the invention. In some embodiments, a cell having increased indentation activity is a cell attempting to penetrate a gel of the invention at least once every 1-2 minutes, once every 1-3 minutes once every 2-4 minutes, once every 3-5 minutes, once every 4-6 minutes, once every 5-7 minutes, once every 6-9 minutes, once every 8-11 minutes, once every 9-14 minutes, once every 10-16 minutes, once every 15-20 minutes, once every 17-35 minutes, once every 20-40 minutes, once every 40-55 minutes, or once every 50-60 minutes. Each possibility represents a separate embodiment of the invention.

In some embodiments, a cell having increased indentation activity is a cell capable of indenting or penetrating a gel of the invention to a depth of at least 1 μm, at least 2 μm, at least 3 μm, at least 4 μm, at least 5 μm, at least 6 μm, at least 7 μm, at least 8 μm, at least 9 μm, at least 10 μm, at least 11 μm, at least 12 μm, at least 13 μm, at least 14 μm, at least 15 μm, at least 16 μm, at least 17 μm, at least 18 μm, at least 19 μm, at least 20 μm, or at least 25 μm or any value and range therebetween. Each possibility represents a separate embodiment of the invention. In some embodiments, a cell having increased indentation activity is a cell capable of indenting or penetrating a gel of the invention to a depth of 0.5-1 μm, 0.7-2 μm, 1-3 μm, 2-4 μm, 3-5 μm, 4-7 μm, 6-9 μm, 8-12 μm, 10-14 μm, 13-17 μm or 16-25 μm. Each possibility represents a separate embodiment of the invention.

In some embodiments, a cell having increased indentation activity is a cell capable of indenting or penetrating a gel of the invention to a depth of at least half the cell's size, at least two thirds of the cell's size, at least the entire cell's size, or at least one and a half times the cell's size. Each possibility represents a separate embodiment of the invention.

In some embodiments, a cell having increased indentation activity is a cell capable of indenting or penetrating a gel of the invention for at least 1 min, at least 5 min, at least 10 min, at least, 20 min, at least 30 min, at least 45 min, at least 60 min, at least 2 hours, at least 3 hours, at least 4 hours, at least 5 hours, at least 6 hours, at least 8 hours, at least 12 hours, at least 15 hours, at least 18 hours, at least 21 hours, or at least 24 hours after the cells were seeded on the gel, or any value and range therebetween. Each possibility represents a separate embodiment of the invention. In some embodiments, a cell having increased indentation activity is a cell capable of indenting or penetrating a gel of the invention for 1-5 min, 4-10 min, 8-20 min, 15-30 min, 25-45 min, 40-60 min, 1-2 hours, 1.5-3 hours, 2-4 hours, 3-5 hours, 5-8 hours, 7-12 hours, 8-15 hours, 14-18 hours, 17-21 hours, or 20-24 hours, after the cells were seeded on the gel. Each possibility represents a separate embodiment of the invention.

In some embodiments, the present invention is directed to methods of diagnosing a disease or condition associated with an increased cell indentation activity in a subject, the methods comprising measuring the indentation activity of a cell from a sample obtained from the subject, wherein increased indentation activity of the cell sample obtained from the subject relative to a control is indicative of a disease or condition associated with an increased cell indentation activity in the subject.

In some embodiments, the methods are directed to determining indentation activity of cells obtained from a subject. In some embodiments, the methods are directed to determining the metastatic potential of cells obtained from a subject. In some embodiments, the methods are directed to predicting the risk of metastases development in a subject. In some embodiments, high metastatic potential correlates with risk of metastases development. In some embodiments, metastatic potential correlates inversely with a subject prognosis. In some embodiments, high metastatic potential is indicative of poor prognosis.

In some embodiments, the methods are directed to cancer diagnosis and prognosis based on a diagnosis/prognosis plot or determination of mechanical invasiveness. In some embodiments, a combined measure of the percentage of indenting cells (%) within a sample, and the cells attained indentation depth (μm), the mechanical invasiveness, is predictive of the metastatic potential of the cells. In some embodiments, the metastatic potential of the cells of a sample indicate the metastatic risk of the sample or the tumor. In some embodiments, the diagnosis/prognosis plot of the present invention provides cutoffs utilized for determining any one of: metastatic potential of cells, risk of metastases development, risk of local invasive spreading into non-tumor sites in an organ, cancer remission, cancer progression cancer-free state, or reduction of invasiveness following treatment, all inferred from cell indentation activity of cells obtained from the subject.

As used herein, the term “non-invasive/benign/normal region” refers to a cutoff utilized in determining a sample of cells as non-invasive, benign or normal, such as exemplified herein in FIG. 4 (dashed box) or FIG. 8B (lower ellipse). In some embodiments, cells having an indentation activity plotted within the non-invasive/benign region cutoff of a diagnosis/prognosis plot, indicate the cells are obtained from a non-invasive/benign/normal origin, such as a subject's tissue or a cell line. In some embodiments, a sample comprising cells plotted within the non-invasive/benign region cutoff, is comprised of 1% at most, 2% at most, 3% at most, 4% at most, 5% at most, 6% at most, 7% at most, 8% at most, 9% at most, 10% at most, 11% at most, 12% at most, 13% at most, 14% at most, 15% at most, 16% at most, 17% at most, 18% at most, 19% at most, 20% at most, 21% at most, 22% at most, 23% at most, 24% at most, or 25% at most indenting cells, or any value and range therebetween, having 0.5 μm at most, 1 μm at most, 1.5 μm at most, 2 μm at most, 2.5 μm at most, 3 μm at most, 3.5 μm at most, 4 μm at most, or 5 μm at most indentation depth, or any value and range therebetween. Each possibility represents a separate embodiment of the invention. In some embodiments, a sample comprising cells falling within the non-invasive/benign region cutoff is comprised of 1-2%, 1.5-3%, 2-4%, 3-5%, 3.5-6%, 4-7%, 5-8%, 5.5-9%, 6.5-10%, 7.5-11%, 8-12%, 9-13%, 10-14%, 11-15%, 12-16%, 13-17%, 14-18%, 15-19%, or 16-20% indenting cells, having 0.5-1 μm, 0.75-1.5 μm, 1.25-2 μm, 1.5-2.5 μm, 2-3 μm, 2.75-3.5 μm, or 3-4 μm indentation depth. Each possibility represents a separate embodiment of the invention. In one embodiment, indentation activity of a cell sample plotted outside the non-invasive/benign box cutoff of the diagnosis/prognosis plot, determines the cell sample was obtained from a cancerous origin such as a tissue of a subject or a cell line.

As used herein below, a high- or low/medium-metastatic potential tumor-origin refer to the likelihood of a tumor to metastasize, i.e., develop metastasis. In some embodiment, high- or low/medium-potential metastatic origin refer to the probability of a tumor to metastasize, i.e., develop metastasis. In one embodiment, cells from a high metastatic potential tumor origin have a high probability to develop metastasis. In one embodiment, a low/medium-potential metastatic origin has a low/medium probability to develop metastasis.

As used herein, the term “invasiveness line”, a non-limiting example of which is given as the top dashed line in FIG. 4, refers to a cutoff utilized in determining the metastatic potential of a cell sample determined to be cancerous (such as given in the top ellipse in FIG. 8B). In some embodiments, the terms “invasiveness line cutoff” and “pre-determined indentation activity threshold” are used herein interchangeably.

In some embodiments, a pre-determined indentation activity threshold is specific to the stiffness or Young's modulus of a gel. In some embodiments, the pre-determined indentation activity threshold is specific to the state of the examined cell, wherein the cell state comprises any one of: a normal cell, a benign cell, a metastatic cancer cell, a high metastatic potential cancer cell, a low metastatic potential cancer cell, a locally invasive cancer cell, a non-metastatic cancer cell, and a pre-cancerous cell. In some embodiments, the pre-determined indentation activity threshold is specific to the tissue or organ origin of the examined cell. Non-limiting examples of a tissue or organ origin includes, but is not limited to, connective tissue, fibrous tissue, bone, muscle, liver, pancreas, blood, among others.

In some embodiments, the pre-determined indentation activity threshold is utilized according to method of the invention, as disclosed herein so as to predict the site of metastases.

In some embodiments, according to the pre-determined indentation activity threshold, reduced indentation activity of at least 5%, at least 10%, at least 20%, at least 35%, at least 50%, at least 75%, at least 90%, or at least 99%, or any value and range therebetween, of a cell population on a first gel compared to the indentation of the cell population on a second gel is predictive of the cell population target organ for metastases is an organ having a Young's modulus being at least 2-fold, at least 3-fold, at least 4-fold, at least 5-fold, at least 6-fold, at least 7-fold, at least 8-fold, at least 9-fold, or at least 10-fold, or any value and range therebetween, greater than the Young's modulus of either the first gel, the second gel, or both. Each possibility represents a separate embodiment of the invention. In some embodiments, according to the pre-determined indentation activity threshold, reduced indentation activity of 5-20%, 7-10%, 15-35%, 30-55%, 50-70%, 65-90%, 85-99%, or 100%, of a cell population on a first gel compared to the indentation of the cell population on a second gel is predictive of the cell population target organ for metastases is an organ having a Young's modulus being 2-4-fold, 3-6-fold, 5-8-fold, 7-10-fold, 8-15-fold, or 10-25-fold, greater than the Young's modulus of either the first gel, the second gel, or both. Each possibility represents a separate embodiment of the invention.

In some embodiments, according to the pre-determined indentation activity threshold, increased indentation activity of at least 5%, at least 10%, at least 20%, at least 35%, at least 50%, at least 75%, at least 90%, or at least 99%, or any value and range therebetween, of a cell population on a first gel compared to the indentation of the cell population on a second gel is predictive of the cell population target organ for metastases is an organ having a Young's modulus being at least 2-fold, at least 3-fold, at least 4-fold, at least 5-fold, at least 6-fold, at least 7-fold, at least 8-fold, at least 9-fold, or at least 10-fold, or any value and range therebetween, greater than the Young's modulus of either the first gel, the second gel, or both. Each possibility represents a separate embodiment of the invention. In some embodiments, according to the pre-determined indentation activity threshold, reduced indentation activity of 5-20%, 7-10%, 15-35%, 30-55%, 50-70%, 65-90%, 85-99%, or 100%, of a cell population on a first compared to the indentation of the cell population on a second gel is predictive of the cell population target organ for metastases is an organ having a Young's modulus being 2-4-fold, 3-6-fold, 5-8-fold, 7-10-fold, 8-15-fold, or 10-25-fold, greater than the Young's modulus of either the first gel, the second gel, or both. Each possibility represents a separate embodiment of the invention.

In some embodiments, cells having an indentation activity plotted above the invasiveness line cutoff of a diagnosis/prognosis plot, indicate the cells are obtained from a high metastatic potential origin, such as a subject's tumor or a cell line. In some embodiments, the invasiveness line cutoff is a linear line. A non-limiting example for a linear invasiveness line on a 2.4 kPa gel fits the equation: y=−2X+60. In one embodiment, a sample comprising cells plotted above the invasiveness line cutoff is comprised of at least 58% indenting cells having at least 1 μm indentation depth. In one embodiment, a sample comprising cells plotted above the invasiveness line cutoff is comprised of at least 56% indenting cells having at least 2 μm indentation depth. In one embodiment, a sample comprising cells plotted above the invasiveness line cutoff is comprised of at least 54% indenting cells having at least 3 μm indentation depth. In one embodiment, a sample comprising cells plotted above the invasiveness line cutoff is comprised of at least 52% indenting cells having at least 4 μm indentation depth. In one embodiment, a sample comprising cells plotted above the invasiveness line cutoff is comprised of at least 50% indenting cells having at least 5 μm indentation depth. In one embodiment, a sample comprising cells plotted above the invasiveness line cutoff is comprised of at least 48% indenting cells having at least 6 μm indentation depth. In one embodiment, a sample comprising cells plotted above the invasiveness line cutoff is comprised of at least 46% indenting cells having at least 7 μm indentation depth. In one embodiment, a sample comprising cells plotted above the invasiveness line cutoff is comprised of at least 44% indenting cells having at least 8 μm indentation depth. In one embodiment, a sample comprising cells plotted above the invasiveness line cutoff is comprised of at least 42% indenting cells having at least 9 μm indentation depth. In one embodiment, a sample comprising cells plotted above the invasiveness line cutoff is comprised of at least 40% indenting cells having at least 10 μm indentation depth. In one embodiment, a sample comprising cells plotted above the invasiveness line cutoff is comprised of at least 38% indenting cells having at least 11 μm indentation depth. In one embodiment, a sample comprising cells plotted above the invasiveness line cutoff is comprised of at least 36% indenting cells having at least 12 μm indentation depth. In one embodiment, a sample comprising cells plotted above the invasiveness line cutoff is comprised of at least 34% indenting cells having at least 13 μm indentation depth. In one embodiment, a sample comprising cells plotted above the invasiveness line cutoff is comprised of at least 32% indenting cells having at least 14 μm indentation depth. In one embodiment, a sample comprising cells plotted above the invasiveness line cutoff is comprised of at least 30% indenting cells having at least 15 μm indentation depth. In one embodiment, a sample comprising cells plotted above the invasiveness line cutoff is comprised of at least 28% indenting cells having at least 16 μm indentation depth. In one embodiment, a sample comprising cells plotted above the invasiveness line cutoff is comprised of at least 26% indenting cells having at least 17 μm indentation depth. In one embodiment, a sample comprising cells plotted above the invasiveness line cutoff is comprised of at least 24% indenting cells having at least 18 μm indentation depth. In one embodiment, a sample comprising cells plotted above the invasiveness line cutoff is comprised of at least 22% indenting cells having at least 19 μm indentation depth. In one embodiment, a sample comprising cells plotted above the invasiveness line cutoff is comprised of at least 20% indenting cells having at least 20 μm indentation depth. In one embodiment, indentation activity of a cell sample plotted below the invasiveness line cutoff of the diagnosis/prognosis plot, determines the cell sample was obtained from a non-highly metastatic origin such as a tissue of a subject or a cell line.

In some embodiments, cells having an indentation activity under the invasiveness line cutoff and above the non-invasive/benign box cutoff of a diagnosis/prognosis plot, indicate the cells are obtained from a low/medium metastatic potential origin, such as a subject's cancerous tumor or a cell line. In one embodiment, a sample comprising cells plotted under the invasiveness line cutoff and above the non-invasive/benign box cutoff is comprised of 20-58% indenting cells having 1 μm at most indentation depth. In one embodiments, a sample comprising cells plotted under the invasiveness line cutoff and above the non-invasive/benign box cutoff is comprised of 20-56% indenting cells having 2 μm at most indentation depth. In one embodiments, a sample comprising cells plotted under the invasiveness line cutoff and above the non-invasive/benign box cutoff is comprised of at least 20-54% indenting cells having 3 μm at most indentation depth. In one embodiments, a sample comprising cells plotted under the invasiveness line cutoff and above the non-invasive/benign box cutoff is comprised of 20-52% indenting cells having 4 μm at most indentation depth. In one embodiments, a sample comprising cells plotted under the invasiveness line cutoff and above the non-invasive/benign box cutoff is comprised of 50% at most indenting cells having 5 μm at most indentation depth. In one embodiments, a sample comprising cells plotted under the invasiveness line cutoff and above the non-invasive/benign box cutoff is comprised of 48% at most indenting cells having 6 μm at most indentation depth. In one embodiments, a sample comprising cells plotted under the invasiveness line cutoff and above the non-invasive/benign box cutoff is comprised of 46% at most indenting cells having 7 μm at most indentation depth. In one embodiments, a sample comprising cells plotted under the invasiveness line cutoff and above the non-invasive/benign box cutoff is comprised of 44% at most indenting cells having 8 μm at most indentation depth. In one embodiments, a sample comprising cells plotted under the invasiveness line cutoff and above the non-invasive/benign box cutoff is comprised of 42% at most indenting cells having 9 μm at most indentation depth. In one embodiments, a sample comprising cells plotted under the invasiveness line cutoff and above the non-invasive/benign box cutoff is comprised of 40% at most indenting cells having 10 μm at most indentation depth. In one embodiments, a sample comprising cells plotted under the invasiveness line cutoff and above the non-invasive/benign box cutoff is comprised of 38% at most indenting cells having 11 μm at most indentation depth. In one embodiments, a sample comprising cells plotted under the invasiveness line cutoff and above the non-invasive/benign box cutoff is comprised of 36% at most indenting cells having 12 μm at most indentation depth. In one embodiments, a sample comprising cells plotted under the invasiveness line cutoff and above the non-invasive/benign box cutoff is comprised of 34% at most indenting cells having 13 μm at most indentation depth. In one embodiment, a sample comprising cells plotted under the invasiveness line cutoff and above the non-invasive/benign box cutoff is comprised of 32% at most indenting cells having 14 μm at most indentation depth. In one embodiments, a sample comprising cells plotted under the invasiveness line cutoff and above the non-invasive/benign box cutoff is comprised of 30% at most indenting cells having 15 μm at most indentation depth. In one embodiments, a sample comprising cells plotted under the invasiveness line cutoff and above the non-invasive/benign box cutoff is comprised of 28% at most indenting cells having 16 μm at most indentation depth. In one embodiments, a sample comprising cells plotted under the invasiveness line cutoff and above the non-invasive/benign box cutoff is comprised of 26% at most indenting cells having 17 μm at most indentation depth. In one embodiments, a sample comprising cells plotted under the invasiveness line cutoff and above the non-invasive/benign box cutoff is comprised of 24% at most indenting cells having 18 μm at most indentation depth. In one embodiments, a sample comprising cells plotted under the invasiveness line cutoff and above the non-invasive/benign box cutoff is comprised of 22% at most indenting cells having 19 μm at most indentation depth. In one embodiments, a sample comprising cells plotted under the invasiveness line cutoff and above the non-invasive/benign box cutoff is comprised of 20% at most indenting cells having 20 μm at most indentation depth.

In some embodiments, a disease or condition associated with an increased cell indentation activity is cancer. In one embodiment, a subject having an increased cell indentation activity is diagnosed with cancer. In one embodiment, a subject having an increased cell indentation activity is afflicted with cancer. In one embodiment, a subject diagnosed with an increased cell indentation activity is afflicted with a locally invasive or a metastatic cancer. In one embodiment, a subject diagnosed with an increased cell indentation activity is predicted to develop local invasion or recurrence of cancer, or afflicted with metastatic cancer. In one embodiment, a subject predicted to have a metastatic cancer has a poor prognosis. In one embodiment, a subject diagnosed with metastatic cancer has a poor prognosis. In one embodiment, poor prognosis is having a survival of about 1%, about 2%, about 3%, about 4%, about 5%, about 6%, about 7%, about 8%, about 9%, about 10%, about 15% or about 25%, at most, or any value and range therebetween. Each possibility represents a separate embodiment of the invention. In one embodiment, poor prognosis is having a survival of 0.5-3%, 1-5%, 2-7%, 3-8%, 4-9%, 5-10%, 6-15%, 7-14%, 8-16%, 9-19%, 10-20%, 12-24%, or 13-25% at most. Each possibility represents a separate embodiment of the invention.

In some embodiments, diagnosis of increased cell indentation in a subject's sample, predicts the likelihood of local invasiveness or recurrence of cancer, or metastases development in the subject. In some embodiments, the likelihood of metastases development in a subject having increased cell indentation activity is at least 50%, at least 60%, at least 70%, at least 80%, at least 90%, or at least 99%, or any value and range therebetween. Each possibility represents a separate embodiment of the invention. In some embodiments, the likelihood of metastases development in a subject having increased cell indentation activity is 40-50%, 45-60%, 55-70%, 65-80%, 70-90%, or 85-100%. Each possibility represents a separate embodiment of the invention.

In some embodiments, an increased cell indentation activity of a subject is increased by at least 5%, at least 10%, at least 20%, at least 40%, at least 60%, at least 80%, at least 100%, at least 120%, at least 140, at least 160%, at least 180%, at least 200%, at least at least 250%, at least 300%, at least 350%, at least 400%, at least 500%, at least 1,000%, at least 2,000%, at least 3,000%, at least 4,000%, or at least 5,000% compared to a baseline level of a control, or any value and range therebetween. Each possibility represents a separate embodiment of the invention. In some embodiments, an increased cell indentation activity of a subject is increased by 1-15%, 10-30%, 25-50%, 45-75%, 70-90%, 80-100%, 90-120%, 100-140%, 130-170%, 150-200%, 190-250%, 225-290%, 275-350%, 340-400%, 380-450%, 425-500%, 475-750%, 500-1,000%, 750-1,500%, 1,000-2,500%, 2,000-3,500%, 3,000-4,500%, or 3,500-5,000% compared to a baseline level of a control. Each possibility represents a separate embodiment of the invention.

In some embodiments, the present invention is directed to methods of excluding a disease or condition associated with an increased cell indentation activity in a subject, comprising measuring the indentation activity of a cell sample obtained from the subject, wherein lower indentation activity of the cell sample obtained from the subject relative to a control is indicative of a lack of disease or condition associated with an increased cell indentation activity in the subject. In some embodiments, a subject having a low cell indentation activity indicates that the cells obtained from the subject are obtained from a tissue selected form the group consisting of: pre-malignant lesion, pre-cancerous tissue, non-cancerous tissue, benign tissue, or normal tissue.

In one embodiment, the present invention is directed to methods of monitoring progression or remission of a disease or condition associated with an increased cell indentation activity in a subject treated against the increased cell indentation activity associated disease or condition, comprising comparing the indentation activity of a cell sample obtained from the subject before and after treatment, wherein reduced indentation activity of a cell sample obtained from the subject after treatment relative to the cell indentation activity of a cell sample obtained from the subject before treatment is indicative of a remissive state of the disease or condition associated with the increased cell indentation activity in the subject. A non-limiting example includes, but is not limited to, a subject identified as having increased cell indentation activity and diagnosed with cancer, treated with chemotherapeutic compounds, irradiation, immunotherapy or any other anti-cancer treatment known to any one of ordinary skill in the art, and then identified as having a lower cell indentation activity, thereby indicating the cells were obtained from a remissive cancerous tissue of the treated subject.

In some embodiments, a low cell indentation activity of a subject is lower by at least 5%, at least 10%, at least 20%, at least 40%, at least 60%, at least 80%, at least 100%, at least 120%, at least 140, at least 160%, at least 180%, at least 200%, at least at least 250%, at least 300%, at least 350%, at least 400%, or at least 500% compared to a baseline level of a control, or any value and range therebetween. Each possibility represents a separate embodiment of the invention. In some embodiments, a low cell indentation activity of a subject is lower by 1-15%, 10-30%, 25-50%, 45-75%, 70-90%, 80-100%, 90-120%, 100-140%, 130-170%, 150-200%, 190-250%, 225-290%, 275-350%, 340-400%, 380-450%, or 425-500% compared to a baseline level of a control. Each possibility represents a separate embodiment of the invention.

In one embodiment, a cell is selected from a non-cancer cell line, a cancer cell line, a malignant cell line, a benign cell line, a metastatic cell line, an immortalized cell line, a naïve cell line, a primary cell culture. In one embodiment, a cell is selected form human-derived cell line or non-human-derived cell line. In one embodiment, a non-cancer cell comprises an immune cell line.

According to some embodiments, methods of the present invention are utilized for a personalized medical diagnosis, prognosis, or treatment of a subject.

As used herein the term “subject” refers to an individual, or a patient, which is a vertebrate, e.g., a mammal, including a human.

As used herein, the term “condition” includes anatomic and physiological deviations from the normal that constitute an impairment of the normal state of the living animal or one of its parts, that interrupts or modifies the performance of the bodily functions.

As defined herein “biological sample” refers to a physical specimen from any animal. In another embodiment, biological sample is obtained from a mammal. In another embodiment, biological sample is obtained from a human. In another embodiment, biological sample is obtained well within the capabilities of those skilled in the art. The biological sample includes, but not limited to, biological fluids such as serum, plasma, vitreous fluid, lymph fluid, synovial fluid, follicular fluid, seminal fluid, amniotic fluid, milk, whole blood, urine, cerebrospinal fluid, saliva, sputum, tears, perspiration, mucus, and tissue culture media, including tissue extracts such as homogenized tissue, and cellular extracts. In another embodiment, a biological sample is a biopsy. In another embodiment, a biological sample is a resected tumor, or any part thereof. In some embodiments, a biological sample is a freshly isolated sample. In another embodiment, a biological sample includes histological sections processed as known by one skilled in the art. The terms “sample” and “biological sample” used herein, are interchangeable.

As used herein, “cancer” encompasses diseases associated with cell proliferation. Non-limiting types of cancer include carcinoma, adenocarcinoma, sarcoma, lymphoma, leukemia, blastoma and germ cells tumors. In one embodiment, carcinoma refers to tumors derived from epithelial cells including but not limited to breast cancer, prostate cancer, lung cancer, pancreas cancer, skin cancer, stomach, liver, and colon cancer. In one embodiment, sarcoma refers of tumors derived from mesenchymal cells including but not limited to sarcoma botryoides, chondrosarcoma, Ewing's sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to Hodgkin lymphoma, non-Hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refer to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, non-germinomatous or non-seminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

### Screening Assays

In another embodiment, the present invention is directed to a method of screening for a compound suitable for preventing cancer invasiveness.

Assays for identification of chemotherapeutic compounds are well known to one skilled in the art and include but are not limited to preparation and screening of chemical combinatorial libraries. Such combinatorial chemical libraries include, but are not limited to, peptide libraries (see, e.g., U.S. Pat. No. 5,010,175, Furka, (1991) Int. J. Pept. Prot. Res. 37: 487-493, Houghton, et al. (1991) Nature 354: 84-88). Peptide synthesis is by no means the only approach envisioned. Other chemistries for generating chemical diversity libraries can also be used. Such chemistries include, but are not limited to; peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs, et al. (1993) Proc. Nat 'I Acad. Sci. USA 90: 6909-6913), vinylogous polypeptides (Hagihara, et al. (1992) J. Amer. Chem. Soc. 114: 6568), nonpeptidal peptidomimetics with a β-D-Glucose scaffolding (Hirschmann, et al., (1992) J Amer. Chem. Soc. 114: 9217-9218), analogous organic syntheses of small compound libraries (Chen, et al. (1994) J. Amer. Chem. Soc. 116: 2661), oligocarbamates (Cho, et al, (1993) Science 261:1303), and/or peptidyl phosphonates (Campbell, et al, (1994) J. Org. Chem. 59: 658; Gordon, et al., (1994) J. Med. Chem. 37: 1385), nucleic acid libraries (see, e.g., Strategene, Corp.), peptide nucleic acid libraries (see, e.g., U.S. Pat. No. 5,539,083) antibody libraries (see, e.g., Vaughn, et al. (1996) Nature Biotechnology 14(3): 309-314), and PCT/US96/10287), carbohydrate libraries (see, e.g., Liang, et al. (1996) Science 274:1520-1522, and U.S. Pat. No. 5,593,853), and small organic molecule libraries (see, e.g., benzodiazepines: Baum (1993) C&EN, January 18, page 33; isoprenoids: U.S. Pat. No. 5,569,588; thiazolidinones and metathiazanones: U.S. Pat. No. 5,549,974; pyrrolidines: U.S. Pat. Nos. 5,525,735 and 5,519,134; morpholino compounds: U.S. Pat. No. 5,506,337; benzodiazepines: 5,288,514; and the like).

In some embodiments, after a library has been created, at least one compound is screened for inhibiting cell invasion. In some embodiments, at least one compound is screened for inhibiting cell indentation. In some embodiments, the inhibitory effect of an assayed compound is calculated by examining the indentation activity of an infiltrating cell is the presence of the assayed compound compared to the indentation activity of an infiltrating cell in the absence of the assayed compound. In some embodiments, the inhibitory effect of an assayed compound is compared to a standard compound. In some embodiments, the indentation activity of an infiltrating cell is the presence of the assayed compound is compared to the indentation activity of an infiltrating cell in the presence of the standard compound. In some embodiments, a standard compound is known to have an anti-cancerous chemotherapeutic activity. Non-limiting examples of which, include, but not limited to, Paclitaxel, Sorafenib and Carboplatin. In some embodiments, the inhibitory effect of an assayed compound is greater than the inhibitory effect of the standard compound. In some embodiments, the inhibitory effect of an assayed compound is comparable to the inhibitory effect of the standard compound. In some embodiments, the inhibitory effect of an assayed compound equals to the inhibitory effect of the standard compound. In some embodiments, the inhibitory effect of an assayed compound is lower than the inhibitory effect of the standard compound.

In some embodiments, the inhibitory effect of an assayed compound over cell indentation is assessed in vitro, ex vivo or in vivo, using one or more gels having one or more stiffnesses or Young's moduli.

In some embodiments, at least one compound is at least 2, at least 3, or at least 4 compounds, or any value and range therebetween. Each possibility represents a separate embodiment of the invention. In some embodiments, at least one compound is 2-3, 2-4, or 3-4 compounds. Each possibility represents a separate embodiment of the invention.

As will be appreciated by one skilled in the art, many types of infiltrating cell lines can be used to screen for a compound having an activity preventing cancer invasiveness, including but not limited to, breast-cancer epithelial cells (e.g., MDA-MB-231 (ATCC HTB-26) and MDA-MB-468 (ATCC HTB-132), lung cancer PC14 cells, colon cancer cell line LoVo, pancreatic cancer cell (ATCC®; TCP-1026), among others.

In some embodiments, a compound capable of reducing the indentation of a cell population is used in treating cancer in a subject in need thereof.

In some embodiments, a compound capable of reducing the indentation of a cell population is used in treating an immune-related disease.

### Computer Program Product

According to some embodiments, there is provided a computer program product for determining cell indentation activity, the computer program product comprising a non-transitory computer-readable storage medium having program instructions embodied therewith, the program instructions executable by at least one hardware processor to: receive measurements of at least one of: (i) an indentation depth or number of indenting cells associated with contacting a cell population with a gel having a Young's modulus of 0.1-20 kPa; or (ii) force applied by the cell population on the gel; and determine a cell characteristic of the cell population based on, at least in part, a pre-determined indentation activity threshold, wherein the cell characteristic is selected from the group consisting of: invasiveness, infiltration, and differentiation state.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

In some embodiments, computer program of the present invention comprises Labview or MATLAB.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

In some embodiments, a computer program of the invention is used for controlling a sensing device. In some embodiments, a sensing device is a microscope, such as, but not limited to a fluorescent microscope, a confocal microscope or others. In some embodiments, a sensing device is a spectrophotometer. In some embodiments, a sensing device is a pH meter. In some embodiments, the sensing device in not a pressure or a strain sensor. In some embodiments, methods and systems of the disclosed invention are directed to a gel and a sensing device for determining cell indentation activity.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, chemical and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Cell Culture

The inventors used various commercially- or otherwise-available, human, epithelial breast and pancreatic cancer and benign cell lines (all from ATCC, Manassas, Va.). For breast cancer cells, the inventors used three cell lines: high metastatic potential (MP, MDA-MB-231) and low MP (MDA-MB-468) breast cancer cells that had been collected from lung metastases, and benign fibrocystic cells (MCF-10A) as control. In addition, the inventors used a high MP breast cancer LM2-4 cell-line that was developed by collecting twice metastasized cells from the human MDA-MB-231 cell line after being seeded in mice. With respect to pancreatic cancer, the inventors used six commercially-available human, pancreatic cell lines: Mia-Paca2 (collected from primary site with no evidence for metastasis), BxPC-3 (collected from primary site with no evidence for metastasis), Panc1 (collected from primary site with one metastasis in lymph nodes), AsPc1 (collected from metastatic site ascites), Capan1 (collected from metastatic site liver), and SW1990 (collected from metastatic site spleen). Cells were cultured in their designated and commonly used media, based on DMEM or RPMI1640 (for all cell lines).

Cells Derived from Fresh Tumors

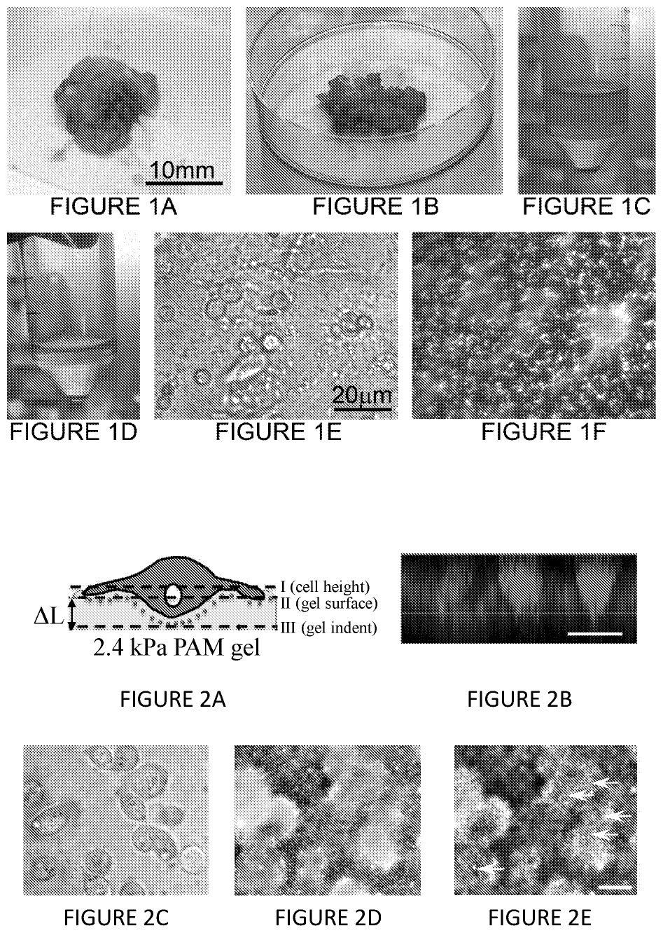

To validate clinical relevance, the inventors performed preliminary testing with pancreatic tumor-samples from human subjects. Tumor samples were provided by the General Surgery Department, Rambam Medical Center at Haifa, Israel (Helsinki approval number: 0285-14). Tumor samples were transported to the lab immediately following surgical removal at 4° C., within the histidine-tryptophan-ketoglutarate (HTK) live preservation solution (Biological Industries, Israel) (Janssen, Janssen, and Broelsch 2003). Sample size was determined in three dimensions by caliper, then weighed, photographically documented and given a running number for archiving purposes. Tumor tissue samples were then processed for cell isolation (FIG. 1). The cells were isolated from minced tissue samples by enzymatic degradation at 37° C. with shaking, using the specialized Tumor Dissociation Kit (Miltenyi Biotec, Auburn, Calif.) in a 2 hours process according to the manufacturers' protocol. The collected cell-extract was passed through 100 μm cell-strainer (Corning Inc., Corning, N.Y.) to separate non-degraded tissue pieces. To remove blood cells and maintain only cells with intact nuclei, the cell samples were concentrated by centrifugation and treatment for 4 min with cell lysis buffer (Roche Diagnostics, Germany). Finally, the cells were transferred into RPMI-1640 cell culture media (Biological Industries, Israel) containing 10% fetal bovine serum (FBS, Hyclone, Mass.), 1% penicillin-streptomycin (Biological Industries, Israel) and immediately seeded on gels for indentation evaluation.

### Polyacrylamide Hydrogel Preparation

The polyacrylamide (PAM) gels were prepared within a range of physiological organ stiffness according to an established protocol (Kristal-Muscal et al., 2013); resulting in gels having Young's modulus of 1,200 Pa, 2,400 Pa and 50,000 Pa. In short, gels were prepared on a 30 mm-diameter, number 5 glass coverslips (Menzel, Germany), with predetermined ratio of the monomers acrylamide, cross-linker BIS-acrylamide (both from Bio-rad, Israel) and water (as specified in Table 1); polymerization was initiated with ammonium persulfate (APS, 0.05% w/v solution) and catalyzed with tertiary aliphatic amine N,N,N′,N′-tetramethylethylenediamine (TEMED, 0.08% v/v, both from Sigma, St Louis, Mo.). Red (excitation/emission 580/605 nm) fluorescent carboxylated polystyrene particles (Molecular probes, Invitrogen life technologies, Carlsbad, Calif.), 200 nm in diameter, were added to gel just below its surface by performing slow gelation at 2° C. under centrifugation at 1,500 rpm for 30 min. Finally, the surface of the gel was activated with Sulfo-SANPAH (Pierce, Thermo Scientific, Waltham, Mass.) 2×10 min under UV light, washed with HEPES buffer, and coated overnight with 5 μg/ml rat tail collagen type I (Sigma, St Louis, Mo.) for cell adhesion.

TABLE 1
Gel composition for example formulations.

| | | Amounts from stock solutions | | | | | | |
|---|---|---|---|---|---|---|---|---|
| | | Acrylamide | | | | | TEMED | |
| | | [μl] 40% | | BIS [μl] 2% | | | [μl] 5% | APS [μl] |
| Young's | | stock (in | | stock (in | | | stock (in | 10% stock |
| modulus | α- | final | | final | | Distilled | final | (in final |
| [kPa] | parameter | solution) | | solution) | | water [μl] | solution) | solution) |
| 1.2 | 0.201 | 26.56 | (4.25%) | 4.59 | (0.037%) | 218.85 | 5 (0.1%) | 1.25 (0.05%) |
| 2.4 | 0.1 | 31.25 | (5%) | 3.13 | (0.025%) | 215.62 | 5 (0.1%) | 1.25 (0.05%) |
| 50 | 0.0045 | 106.25 | (17%) | 1.65 | (0.013) | 142.1 | 5 (0.1%) | 1.25 (0.05%) |

Using a rheometer, the inventors obtained the average Shear modulus and from that the Young's modulus of the gels. The shear modulus (G*) of the gels is determined using TA Instruments AR-G2 rheometer using a 2-cm parallel plate fixture (New Castle, USA). The complex shear modulus, G*, was effectively equal to the elastic modulus G′, indicating an elastic gel material. Thus, the inventors were able to calculate the Young's modulus, E, through the following relation: E=2G*(1+v), which for the PAM gel, with a typical Poisson's ratio, v, of 0.49 (Boudou et al., 2009) becomes E=3G′.

### Microscopic Imaging and Indentation Depth Determination

The inventors seeded 3×10⁵ cells on each gel within the respective media, resulting in an average of 25±5 cells per field-of-view (area of 0.016 mm²). The seeded cells were adjacent and/or touching, and typically remained in a monolayer without overlapping. The imaging was done with an inverted, epifluorescence Olympus IX81 microscope, using a 60×/0.7 numerical aperture (NA) differential interference contrast (DIC, Nomarsky optics) air-immersion, long working-distance objective lens. The cells were maintained in 37° C., 5% CO₂, and high humidity (90%) throughout the entire experiment to sustain their viability. Imaging and indentation depth measurements were initiated approximately 45 minutes after seeding. In each gel the inventors randomly documented 9-10 fields-of-view. The focal depth of each image was recorded independently during the experiment, following manual focusing, using an automated, computer-controlled microscope stage. For each cell line the inventors have repeated the experiments at least 3 times and in 3 triplicates, resulting in hundreds of imaged indenting and non-indenting cells.

At each measurement time-point, locations on the gel were randomly chosen and at least 3 images were taken: (i) a DIC image of the cells on the gel, (ii) a fluorescence image of the particles embedded at the focal plane of the gel surface, and (iii) a series of fluorescence images at the lowest focal depth where particles were observed, to identify each indenting cells' depth. The inventors imaged typically 5-6 focal depths below the gel height, where 1-8 indenting cells were in focus at each depth (FIG. 2Figs). The indentation depth was then calculated by the difference in focal depths between the fluorescence image at the gel surface (undisturbed gel) and at the lowest focal plane where particles are in focus, i.e., at the bottom of the specific indenting cell. Images were analyzed using a custom-designed module (Kristal-Muscal et al., 2013) in MATLAB 2012b (The Mathworks, Nattick, Mass.) to determine the number of viable and indenting cells, as well as the indentation depth of each cell; the inventors determined the number of indenting cells out of the total adhered cells.

### Confocal Imaging

To demonstrate that the changes in focal depth of the particles (embedded in the gel surface) correlate to indentations caused by cells, the inventors provided confocal images and side views of the gels with indenting cells (FIG. 2B). Cells were seeded on gels with red-fluorescent particles and incubated for 1 hour. Following that, the cells were fixated with 3.2% (v/v) Paraformaldehyde (PFA, Electron Microscopy Sciences, Hatfield, Pa.), then permeabilized with 0.5% (v/v) AR-grade Triton X-100 (Bio Lab, Israel), and blocked with 3% (v/v) FBS (Hyclone, ThermoFisher Scientific, Waltham, Mass.). The nuclei of the fixated cells were stained using Hoechst 33342 (Sigma, St Louis, Mo.). Cells were imaged with a spectral-imaging Zeiss LSM700 confocal system, mounted on a motorized Axio Observer Z1 microscope, using a 20×/0.4NA objective lens. Images were taken in stacks of 12-14 slices in z-scale distance of 3 μm.

### Cell Viability Staining

Calcein-AM fluorometric assay (BioVision, USA) was used for viability staining (Weston and Parish 1990) and was performed on all cell samples. Hydrolysis of Calcein AM by intracellular esterase produced a hydrophilic, strongly fluorescent compound that was retained in the cell cytoplasm and was measured (Excitation/Emission=485 nm/530 nm) 30 min after staining. Hoechst 33342 (Sigma, St. Louis, Mo.) was used for nucleus staining when the enzymatic reaction resulted in fluorescent stain of the nuclear after 1.5-2 hours of incubation. The number of viable cells out of the total number of cells on the gel was determined by overlay of the Calcein and Hoechst stains, being at over 90% for all samples.

### Chemotherapeutic Treatments

Paclitaxel (Taxol, Cytoskeleton Ltd., Denver, Colo.) was added at concentration of 25 μM to the cells attached to the gels; a stock solution of 0.01 M in Dimethyl Sulphoxide (DMSO, Sigma, St Louis, Mo.) was diluted with cell-growth media. The cells were incubated with the drug for 1-2 hours before imaging. At least three independent experiments were performed with each cell line and compared to untreated control.

### Finite Element Analysis and Simulation Model

The inventors have performed a finite element (FE) analysis using the FE Bio Software Suite (Version 2.6.4, Scientific Computing and Imaging Institute, University of Utah, UT) to simulate the effects of indenting objects (cells) on the gel. The inventors have simulated a simplified system including multiple, three-dimensional cylinders that indent the gel to the average indentation depth measured in cells, i.e. 6 μm. The cylinders were 12 μm in diameter and 20 μm in height and were defined as neo-Hookean material with a Young's modulus of 25 kPa and Poisson's ratio of 0.49; these have previously been used as representative measures for various types of cells (Calzado-Martin et al., 2016). The gel was simulated as a neo-Hookean material with Young's modulus of 2.4 kPa and Poisson's ratio of 0.49. The inventors defined the gel as a box structure with surface area 300×300 μm² and height of 100 μm. A group of 9 cylinders were placed in a small location on the gel, and the inventors evaluated the stress and strain in a small region (120×120×8 μm³) at the bottom of the gel underneath the cells.

### Example 1

### Formulating Polyacrylamide Hydrogels

The inventors have identified specific formulations of the polyacrylamide (PAM) gels that result in a stiffness and structure that facilitates indentations caused by invasive subsets of cells. Those formulations are well defined by a specific correlation of the concentrations (as v/v or %) of the acrylamide ([ACR]), the BIS-acrylamide monomer/cross-linker ([BIS]) and the overall volume of the polymerizing solution in the following way:

$$α = \frac{C}{T ( 1 - C )} \qquad Equation 1$$

Where the parameters T and C (in different contexts) have typically been used to describe gel stiffness correlations.

$$\begin{matrix}T = \frac{[ BIS ] + [ A C R ]}{Total volume} & Equation 2 \\ C = \frac{[ BIS ]}{[ BIS ] + [ A C R ]} & Equation 3\end{matrix}$$

To understand the difference between gels of the present invention (allowing to see and measure cell-indentation) and gels used by others, the inventors have analyzed gel-formulations (see example 5).

For the specific gel-formulations of the present invention, the inventors introduced a new parameter “α” (Equation 1) through a unique combination of the “C” and “T” parameters.

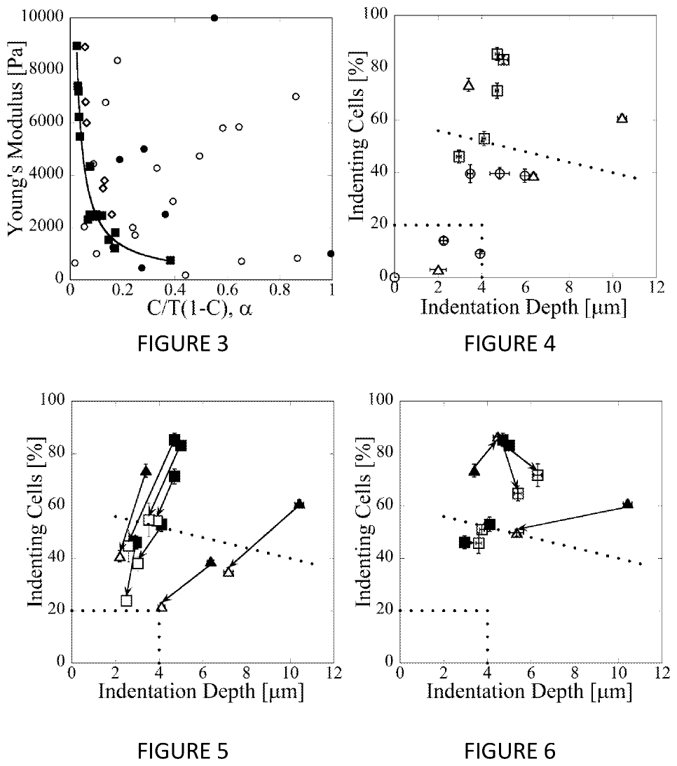

The inventors have analyzed in more than 15 studies different stiffness PAM gels and have found that 10 of them linearly (or partially linearly) fitted to parameter α (FIG. 3).

### Example 2

### Cell Indentation Studies

Cells were seeded and allowed to adhere for 45 minutes, and then for up to 6 more hours, when the number of indenting cells and the indentation depths attained by the groups of closely situated, non-aggregated cells were evaluated. A variety of human breast and pancreatic cells from cell-lines as well as cells extracted from human pancreatic tissues were evaluated and each exhibited different number of indenting cells and attained depths (FIG. 4). Cell lines were run at least on 3 different days with at least 2 repeats each, and with 10-30 random fields of view imaged and averaged for each gel. Tumor samples were run when fresh, on at least one gel and at least 20 random fields of view imaged and averaged.

Benign cell lines from breast or normal cells or pre-malignant samples from healthy tissue adjacent to a pancreatic tumor did not indent the gels or indented in smaller amounts and to lower depths. Specifically, in benign (cell lines and patient samples) or clinically diagnosed pre-malignant samples, a small percentage (<20%) of cells indent the gels and those attained lower depths (<4 μm); the cell line results matched the fresh samples and provide a cutoff for benign/cancer diagnosis (FIG. 4).

The inventors observed that cells with high metastatic potential (large invasiveness) achieved deeper indentation depths and a larger percentage of the cells indented the gels (FIG. 4). Using the results of the established cell lines from breast and pancreatic cancers, with predetermined (and verified by Boyden chamber assay; FIG. 8E) high/low metastatic potential, the inventors defined a range of cutoffs to distinguish high and low metastatic potential cells. The tumor samples were then assessed according to this prognostic measure and verified with clinical, histopathological prognosis and eventual clinical, long-term outcomes in patients when available. The indentation capacity and activity of cells freshly harvested from human-subject tissue samples correlated to clinical diagnosis and with clinical outcome (in cases of high metastatic potential the metastasis development in patients was rapid). Using the results from the established cell lines combined with the results obtained from the clinically determined fresh tissue samples, the inventors have specified cutoffs for benign/non-invasive cells and distinguishing between cells with high and low metastatic potential, therefore providing diagnostic and prognostic testes—termed a diagnosis/prognosis plot.

The diagnostic and prognostic cutoffs were strengthened by further experiments, applying chemotherapeutic drugs, which also showed the applicability of the gel-platform as a drug response testing-platform. The inventors applied the chemotherapeutic drug Taxol (25 μm for 1-2 hr) to breast and pancreatic cancer cell lines and observed that the indentation capacity was reduced in all cells, i.e. reduction in percentage of indenting cells as well as attained indentation depths (FIG. 5). Interestingly, all invasive cell lines with either high or low metastatic potential moved from their previous location (as plotted in FIG. 4) under or closer to the nearest cutoff, i.e., invasive cells moved below the invasiveness line and less invasive cells moved closer to the non-invasive/benign region box; possibly indicating a reduction of invasiveness and metastatic risk of these cells.

The inventors evaluated the effect of gel stiffness on the ability to accurately identify and distinguish invasive subpopulations of breast cancer versus pancreatic cancer. The evaluated cell-lines exhibit different responses to substrate stiffness in terms of the percentage of indenting cells and indentation depths (FIG. 6). The inventors have initially used 2.4 kPa gels for all cell types. Softer gels (1.2 kPa) have improved the resolution and thus the deliverable prognosis (invasive capacity) as shown for highly metastatic pancreatic cell lines; the inventors were able to distinguish differences between pancreatic cancer cells with higher and lower metastatic potential and from different metastatic sites with more accuracy. Moreover, using lower stiffness gels had little effect on benign/non-invasive cells and maintained the same diagnostic result. In contrast, for the tested breast cancer cell lines, the softer gel appeared less applicable. Specifically, the inventors showed for example that one of the most invasive breast cancer cell lines moved down in predicted prognosis, to the low/high MP cutoff invasiveness line (FIG. 6). Thus, for different (suspected) cancer types, a combination of different substrate stiffness is likely be applied for rapid and accurate cancer diagnosis and metastases prognosis.

### Example 3

### Estimation and Modeling of Gel Response and Exemplary Calculation

The inventors used a single cell forces to estimate the forces that would be applied by several cells seeded closely on the gel; as adjacent cells may synergistically interact and induce larger indentations and forces. The inventors observed that of 300,000 cells that were seeded on a gel (10×10 mm²) about 80% on average were attached to the gel surface (regardless of a cell type). Of the attached cells, only partial percentage indented the gel, i.e. corresponding to their metastatic potential (MP). Assuming 35% of attached cells are low MP and thus indent the gel (as was shown for breast cancer; Alvarez-Elizondo and Weihs 2017), approximately 84,000 indenting cells are calculated with a total force of 0.039 N applied to a gel surface, which when homogeneously distributed over the entire gel is providing a pressure of 0.39 kPa.

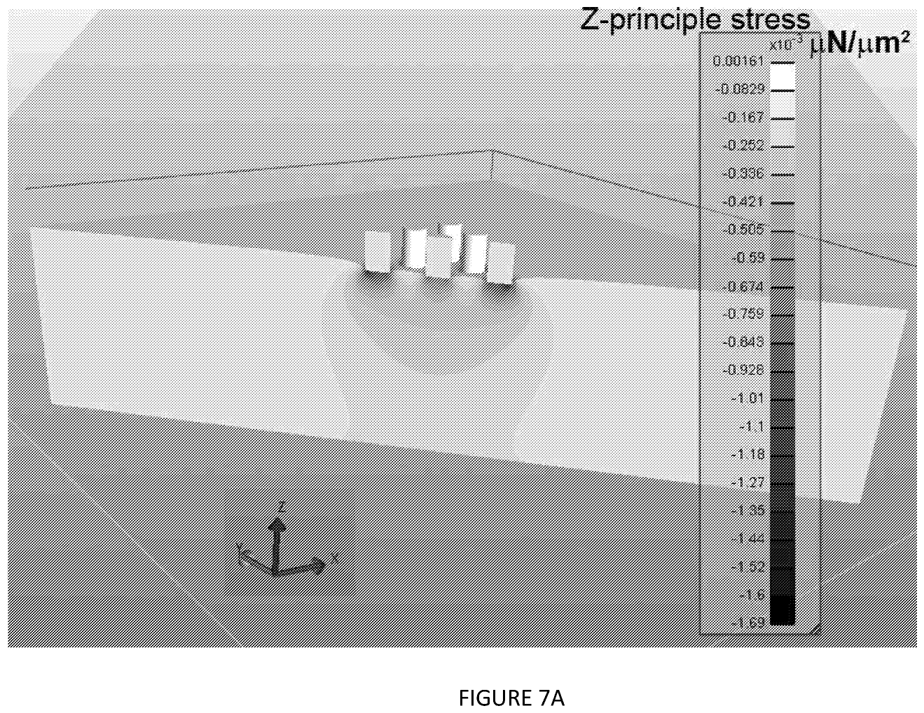

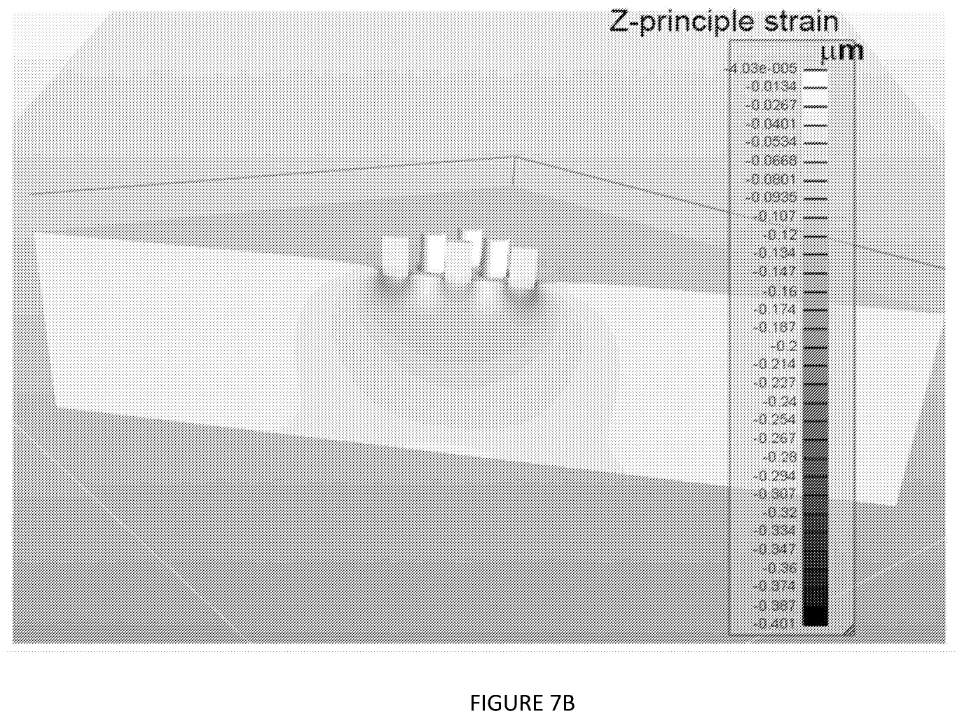

The inventors further evaluated the effects of cell indentation (simulated as indenting rods) on gels by FE simulations. The inventors observed that the elastic gel transmits significant and measurable stresses and strains to depths of 100 μm below the gel's surface level (FIGS. 7A-B).

### Example 4

### Mechanical Invasiveness

The inventors have solidified the rapid (2-hr) diagnosis and prognosis capacity of the mechanical invasiveness measure (the combined percent indenting cells and their attained depths) in terms of cancer stratification and potential classification. The inventors have specifically conducted additional clinical studies, showing that the cell indentation technology worked in pancreatic, stomach and non-melanoma skin cancers (FIGS. 8A and 8C), providing rapid classification and metastasis prediction, which coincided with the clinical histopathology that was available only weeks after the clinical intervention and the inventors' prompt diagnostic and/or prognostic measurement. In one important case of an invasive squamous cell carcinoma (SCC skin cancer), the indentation technology was uniquely able to sensitively identify what the physicians classify as “active cells” or in situ cancer that is limited to the epidermis (FIG. 8C); Based on this, collaborating physicians requested the inventors to test different sections in the skin separately to corroborate their clinical prognosis.

The inventors have further developed the technology so that beyond the diagnosis of cancer (yes/no) and prognosis (likelihood for metastasis) it can very uniquely indicate highly probable site for metastasis formation (FIG. 8D). Specifically, the inventors have developed a measure based on comparing the indentation capacity, or mechanical invasiveness through the percent of cells indenting differentially on two gels with different stiffness, for example a soft (1.2 kPa) gel vs. a stiffer (2.4 kPa) gel. Mechanical invasiveness of non-metastatic cancer cells was low and unaffected by the gel stiffness. In contrast, metastatic cells demonstrated two different responses, either having more cells indent on the softer gels or on the stiffer gels, those correlated with the cell lines' metastatic site being, respectively, in a soft or stiff organ.

### Example 5

### Correct Evaluation of Gel Stiffness

The inventors have highlighted differences in the compositions and the stiffness of the gel of the invention, as compared to gels used in the literature. The inventors have measured and compared the stiffness of the gel of the invention with the formulations described by Kraning-Rush et al., (2012) and by Mierke et al., (2011) using rheometry as a gold-standard for stiffness measurement. Significant differences were observed for the same compositions examined under rheometry and ball indentation methodology, which are summarized hereinbelow (Table 2).

TABLE 2
Comparison of gel stiffness measurement
method in different PAM gel compositions

| | Reported | | | |
|---|---|---|---|---|
| | gel | | | Actual |
| | stiffness | w/v % | w/v % BIS- | stiffness |
| Gel | [Pa] | Acrylamide | acrylamide | (Rheometry)^{b} |
| Mierke et al., | 5,400 ^{c} | 4.7 | 0.24 | — |
| (2011) | | | | |
| Kraning-Rush | 5,000 ^{a} | 7.5 | 0.19 | 20,800 |
| et al., (2012) | | | | |
| Gel of the | 5,000 | 7.5 | 0.027 | 5,025 |
| invention | | | | |
| Kraning-Rush | 2,500 ^{a} | 5 | 0.1 | 6,640 |
| et al., (2012) | | | | |
| Gel of the | 2,400 | 5.72 | 0.032 | 2,400 |
| invention | | | | |
| Kraning-Rush | 1,000 ^{a} | 1.5 | 0.1 | 1,045 |
| et al., (2012) | | | | |
| Gel of the | 1,200 | 4.25 | 0.037 | 1,200 |
| invention | | | | |

^{a} Gel stiffness measured by ball-indentation method using steel ball with 0.32 mm radius (Abbott Ball Co., West Hartford, CT) placed on a gel with embedded fluorescent beads and determining indentation with phase contrast imaging 20× magnification as described (Kraning-Rush et al., 2012).
^{b}Gel rheology measured using TA Instruments AR-G2 rheometer (New Castle, DE).
^{c} Gel stiffness measured from the linear extension of a cylinder of gel (16-mm diameter, 50-mm length) under force as described (Fabry et al., 2011).

In order to compare the inventors' experimental results with previously reported results Kraning-Rush et al. (2012), gels were prepared according to procedures and compositions described Kraning-Rush et al., (2012), yet stiffness was verified using the rheometer. While the obtained values for the 1 kPa gels were similar, significantly higher values were observed in all other tested formulations as compared to reported values measured by ball indentation (FIG. 9). The difference between the rheometry and ball-indentation techniques increased with the gel stiffness. This aspect is crucial especially in the context of the invention disclosed herein, as the inventors have demonstrated that low stiffness gels are required to determine indentation activity and further a cell characteristic, such as invasiveness, metastasis potential, infiltration, etc.

### Example 6

### Microscopy Magnification Effects

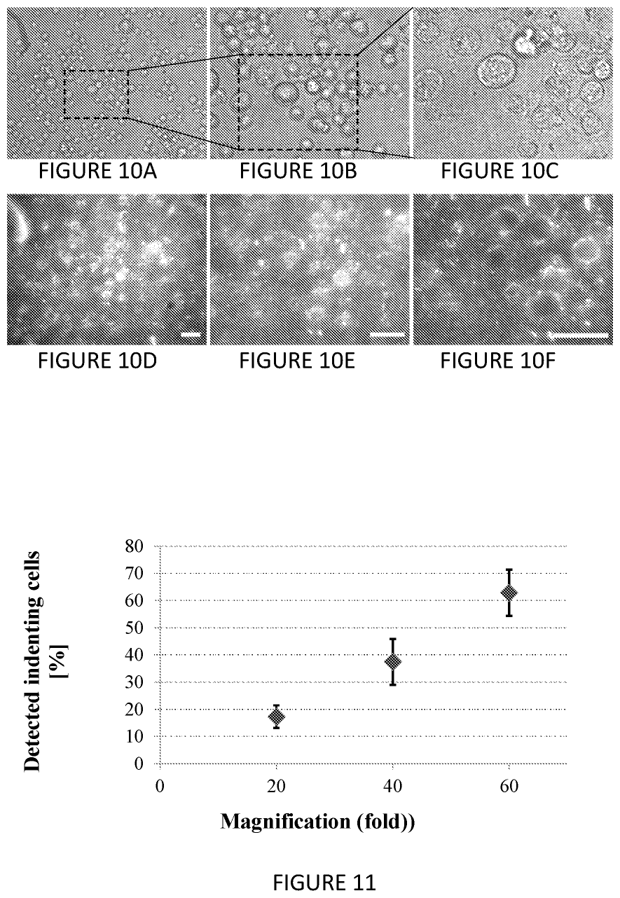

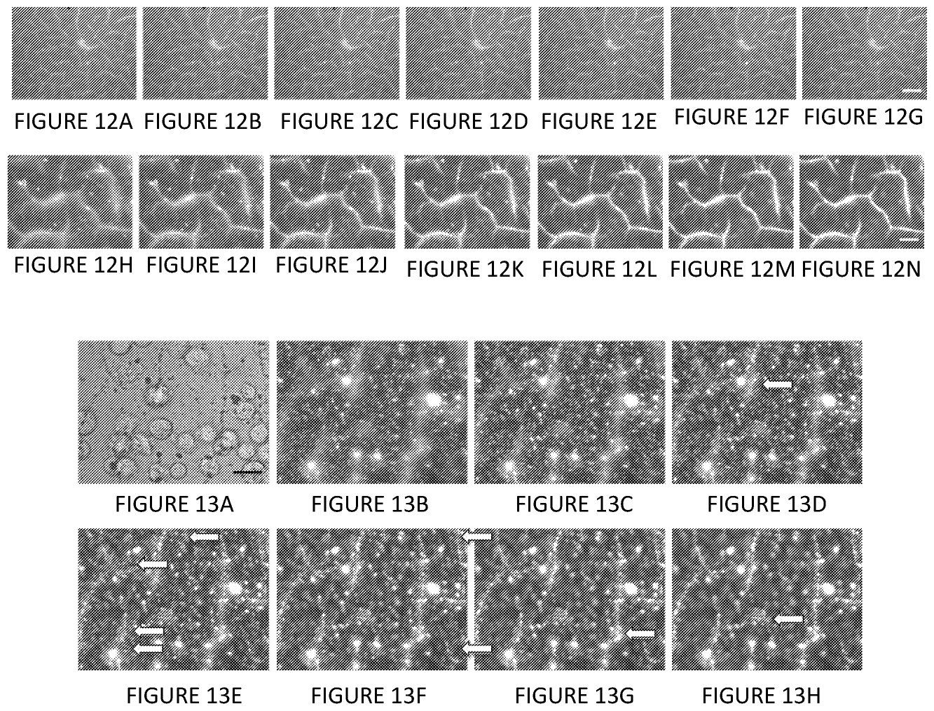

The inventors showed that the specific magnification during imaging of cells on gels appear was a critical factor for the identification of the indenting cells and calculating the indentation depths. In contrast to traction force microscopy (TFM) experiments, in which magnification is typically 20× (20×/0.5 NA phase contrast dry objective) or 40× (40×/0.6 NA phase contrast objective) for detecting lateral force, according to the disclosed invention, magnification of 60× (e.g., 60×/0.7NA differential interference contrast, air-immersion) is required for indentation measurements. To evaluate the role of magnification on the indentation assay—the same random fields of view, on a 1.2 kPa PAM with indenting MDA-MB-231 (high MP breast cancer cells), were imaged under different magnification (FIG. 10). It was clearly observable that under lower magnification, less indenting cells can be identified, even with an experienced user searching for them. That is, the percentage of detected indenting cells significantly reduced with reduction in magnification (FIG. 11). Furthermore, the indentation depth could not be accurately determined at low magnification, even when the focal height was controlled by an automated microscope stage (FIGS. 12A-12G); images from different focal heights and low magnification were indistinguishable (by eye) even with 6 μm difference. In contrast, in images obtained at ×60 magnification (FIGS. 12H-12N) with manual focusing using the delicate setting in the microscope (as per standard procedure) the different focal depths were clearly distinguishable.

### Example 7

### Effects of Particle Size in Generated Gels

The inventors showed that the size of the fluorescent particle embedded at the gel surface affects the resolution of the imaged interaction of the cells with the gel. Specifically, it was shown that smaller particles provided higher resolution in terms of deformation within smaller regions. Furthermore, this increased resolution required higher magnification imaging, as demonstrated herein above. The inventors have compared 2.4 kPa PAM gels embedded with either 200 nm or 500 nm red fluorescent beads. Both particle sizes were image under high magnification (×60), according to manual optimal focus determination.

Initially the inventors have determined the resolution of depth that may be accurately attained using a microscope system under each condition, i.e. the error in depth determination due to different focal depths where particles appear to be in correct focus (meaning, the error in the identification of the correct focal plane of the fluorescent beads). Under the sample experimental conditions (2.4 kPa gels), the inventors have determined a vertical resolution of 0.9±0.3 μm and 1.8±0.7 μm in localization of 200 nm and 500 nm beads, respectively. Moreover, when gels embedded with 500 nm beads were imaged at lower magnification of ×40 or ×20, the resolution had reduced, leading to a larger focal depth uncertainty of 4.2±2 μm and 3.7±2 μm (p=0.21), respectively. Thus, under low magnification and large (500 nm) beads, indentation depths of less than 4 μm are statistically indistinguishable (on this specific gel stiffness). As noted, particles are typically seeded in high density. The inventors showed that it is the particle size that determines the smallest gel-surface vertical deformations (indentations) that can be observed. If particles are to be seeded in lower density, the resolution would reduce even further.

To verify the effect of fluorescent bead size on the cell indentation assay, the inventors have performed experiments utilizing high MP breast cancer cells on a 2.4 kPa PAM. The inventors have found, that even with highly invasive cells seeded at high density it was more difficult to identify indenting cells when the fluorescent beads were larger (FIG. 13). In gels embedded with either 500 nm or 200 nm beads, the percent of indenting cells that were identified was 35±15% or 73±12%, respectively. That is, the highly invasive cells appeared to indent in amounts similar to low metastatic potential cells. It is important to note that such differences would make differential diagnosis/prognosis unreliable and irreproducible since differences between cancer/metastatic cells would become smaller or even insignificant, therefore rendering the indentation assay unfeasible. Moreover, using imaging at low magnification ×20 of 2.4 kPa gel embedded with 500 nm beads, the inventors were only able to recognize as little as 7.4±4% of indenting cells. As this experiment repeated the set-up of other research groups, e.g., Bordeleau et al., (2016), it may strengthen the hypothesis why indentations were not observed and or recorded by others. Based on the aforementioned, it is the combination of high-magnification and small fiducial-marker bead size are critical to facilitate the high-resolution, accurate, and reproducible gel indentation assay.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. (canceled)

2. A method of classifying a cell population according to indentation activity, the method comprising:
a. contacting a cell population with a first gel having a Young's modulus of 0.1-20 kPa;
b. measuring a cell indentation parameter, thereby determining said cell population indentation activity; and
c. determining a cell characteristic of said cell population based on a pre-determined indentation activity threshold, wherein said cell characteristic is selected from the group consisting of: invasiveness, metastatic potential, infiltration, and differentiation state,
thereby classifying the cell population according to the indentation activity.

3. The method of claim 2, wherein said indentation activity parameter comprises the number of indenting cells, the indentation depth attained by said cells, the force applied by said cells to said gel, the pressure applied by said cells to said gel, the strain applied by said cells to said gel, the displacement applied by said cells to said gel, or any combination thereof.

4. The method of claim 2, wherein said cell population is obtained from a sample being obtained from a subject.

5. The method of claim 2, for diagnosing cancer in a subject, wherein increased indentation activity of said cell population relative to control is indicative of cancer in said subject.

6. The method of claim 5, further comprising a step of quantifying said cell population indentation activity, wherein increased indentation activity of said cell population relative to control is a prediction or prognosis of metastatic cancer in said subject.

7. The method of claim 6, wherein said prediction of said metastatic cancer comprises predicting the target organ for metastases by further comparing the indentation activity of said cell population on a second gel having a different stiffness compared to said first gel.

8. The method of claim 2, for screening for a compound suitable for reducing indentation activity of said cell population, the method comprising contacting the cell population with said compound, wherein reduction of indentation activity of said cell population in the presence of said compound compared to the indentation activity of said cell population in the absence of said compound indicates said compound is suitable for reducing indentation activity of said cell population.

9. The method of claim 8, wherein said cell population is contacted with said compound prior to contact with the gel, after contact with the gel, or both.

10. The method of claim 8, wherein said compound suitable for reducing indentation activity of a cell being suitable for preventing or reducing cancer invasiveness.

11. The method of claim 2, wherein said measuring comprises the use of a sensor, wherein said sensor is selected from the group consisting of: a pressure sensor, a strain sensor, and an optical sensor.

12. The method of claim 2, wherein said gel further comprises particles, and optionally wherein: (a) said particles are fluorescent particles; (b) said particles are 10 nm to 450 nm in diameter; or both.

13. (canceled)

14. (canceled)

15. The method of claim 2, wherein said cell is an infiltrating cell, and optionally wherein any one of: (a) said infiltrating cell is a proliferating cell; (b) said proliferating cell is a cancer cell; (c) said cancer cell is a metastatic cancer cell; and (d) said cancer cell is a locally invasive cancer cell.

16. (canceled)

17. (canceled)

18. (canceled)

19. (canceled)

20. A computer program product for determining cell indentation activity, the computer program product comprising a non-transitory computer-readable storage medium having program instructions embodied therewith, the program instructions executable by at least one hardware processor to:
a. receive measurements of at least one cell indentation parameter of a cell population contacted with a gel having a Young's modulus of 0.1-20 kPa; and
b. determine a cell characteristic of said cell population based on, at least in part, a pre-determined indentation activity threshold, wherein said cell characteristic is selected from the group consisting of: invasiveness, metastatic potential, infiltration, and differentiation state.

21. The computer program product of claim 20, wherein said indentation activity parameter comprises the number of indenting cells, the indentation depth attained by said cells, the force applied by said cells to said gel, the pressure applied by said cells to said gel, the strain applied by said cells to said gel, the displacement applied by said cells to said gel, or any combination thereof.

22. The computer program product of claim 20, wherein said cell population is obtained from a sample being obtained from a subject, and optionally wherein increased indentation activity of said cell population relative to control is an indication of cancer, or a prediction or prognosis of metastatic cancer, in said subject.

23. (canceled)

24. A device comprising:
a. a gel having a Young's modulus of 0.1-20 kPa; and
b. at least one sensor responsive to signals ranging between 1 mPa-20 kPa, in contact with said gel.

25. The device of claim 24, wherein said gel is a hydrogel comprising at least 50% water by weight, and optionally said gel comprises at least one biologically inert polymer.

26. (canceled)

27. The device of claim 24, wherein said gel is impenetrable to a cell, and optionally wherein said gel comprises pores, wherein at least 80% of said pores have a diameter of between 1 and 500 nm.

28. (canceled)

29. The device of claim 24, wherein said gel has a thickness of 30-500 μm.

30. The device of claim 24, wherein said sensor is selected from a pressure sensor or a strain sensor.

31. The device of claim 24, further comprising an optical sensor.
